(19) United States
(12) Patent Application Publication
(10) Pub. No.: US 2011/0250129 A1
(43) Pub. Date: Oct. 13, 2011

(54) BIOLUMINESCENCE IMAGING-BASED SCREENING ASSAY AND INHIBITORS OF ABCG2

(75) Inventors: Martin Gilbert Pomper, Baltimore, MD (US); Yimao Zhang, Baltimore, MD (US); John Laterra, Baltimore, MD (US)

(73) Assignee: The Johns Hopkins University, Baltimore, MD (US)

(21) Appl. No.: 13/129,037

(22) PCT Filed: Nov. 12, 2009
(86) PCT No.: PCT/US09/64200

§ 371 Date: Jun. 27, 2011

Related U.S. Application Data
(60) Provisional application No. 61/113,723, filed on Nov. 12, 2008.
(60) Provisional application No. 61/175,994, filed on May 6, 2009.

(51) Int. Cl. A61K 51/00; C12Q 1/02; A61K 36/752; A61K 31/423; A61P 35/00; A61K 31/409; A61K 31/453; A61K 31/435; A61K 31/5415; A61P 25/00; C12Q 1/66; A61K 31/517

(52) U.S. Cl. 424/1.11; 435/8; 435/29; 424/736; 514/375; 514/252.17; 514/410; 514/320; 514/297; 514/225.5

(57) ABSTRACT

A bioluminescence imaging-based high-throughput assay for inhibitors of ABCG2 is described. Compositions of inhibitors of ABCG2 and methods of using ABCG2 inhibitors are also described.

### CROSS-REFERENCE TO RELATED APPLICATION

This application claims priority to U.S. Provisional Application No. 61/113,723 filed Nov. 12, 2008 and U.S. Provisional Application No. 61/175,994, filed May 6, 2009, the entire contents of which are hereby incorporated by reference. This invention was made using U.S. Government support under NIH grant U24 CA92871. The government has certain rights in this invention.

### BACKGROUND

1. Field of the Invention

The present invention relates to inhibitors of ABCG2, a member of the ATP binding cassette (ABC) family of transporters and a bioluminescence imaging-based high-throughput screening assay for identifying inhibitors of ABCG2.

2. Background of the Invention

ABCG2 is a recently described member of the ATP-binding cassette (ABC) transporters, a family of proteins that use the energy of ATP hydrolysis to transport certain chemicals out of cells (Doyle et al., “A multidrug resistance transporter from human MCF-7 breast cancer cells,” Proceedings of the National Academy of Sciences of the United States of America, vol. 95, no. 26, pp. 15665-15670, 1998; Szakacs et al., “Targeting multidrug resistance in cancer, ” Nature reviews, vol. 5, no. 3, pp. 219-234, 2006). The overexpression of ABC transporters has been associated with multidrug resistance (MDR), a major impediment to successful cancer chemotherapy. ABCG2 confers resistance to several chemotherapeutic agents such as mitoxantrone (MTX), daunorubicin, doxorubicin, bisantrene, topotecan and flavopiridol (Benderra et al., “Breast cancer resistance protein and P-glycoprotein in 149 adult acute myeloid leukemias,” Clin Cancer Res, vol. 10, no. 23, pp. 7896-7902, 2004). Previously, it has been reported that ABCG2 is expressed in the brain, the colon, small and large intestine, venuous endothelium, and in capillaries, and it was thought that the expression pattern indicates that ABCG2 plays a protective in these tissues although further evidence was needed to support this hypothesis (Robey et al., “ABCG2: determining its relevance in clinical drug resistance,” Cancer metastasis reviews, vol. 26, no. 1, pp. 39-57, 2007). In addition, ABCG2 has been found to affect drug transport across the gastrointestinal epithelium and blood-brain barrier (Robey et al., “ABCG2: determining its relevance in clinical drug resistance,” Cancer metastasis reviews, vol. 26, no. 1, pp. 39-57, 2007). Many believe that judiciously combing ABCG2 inhibitor(s) with standard cancer chemotherapy will nullify the protection tumor cells receive, preventing cancer survival and metastasis (Robey et al., “ABCG2: determining its relevance in clinical drug resistance,” Cancer metastasis reviews, vol. 26, no. 1, pp. 39-57, 2007; Ailles et al., “Cancer stem cells in solid tumors,” Current opinion in biotechnology, vol. 18, no. 5, pp. 460-466, 2007; Szakacs et al., “Targeting multidrug resistance in cancer, ” Nature reviews, vol. 5, no. 3, pp. 219-234, 2006). However, this idea remains to be tested, largely due to the lack of suitable ABCG2 inhibitors, despite significant efforts at uncovering them.

### SUMMARY

The present invention includes methods for identifying inhibitors of ABCG2 by imaging at least one culture comprising a test compound, luciferin and cells that express ABCG2 and firefly luciferase and selecting a test compound resulting in at least two-fold bioluminescence signal enchancement as an ABCG2 inhibitor.

Embodiments include compositions and methods for treating a cellular proliferative disorder by administering a ABCG2 inhibitor identified by the described method to a patient in need of treatment. In some embodiments, the ABCG2 inhibitor is administered in combination with a chemotherapeutic agent different from the ABCG2 inhibitor.

Examples of a cellular proliferative disorder include, but are not limited to, acute myelogenous leukemia, acute lymphoblastic leukemia, multiple myeloma, non-Hodgkin's lymphoma, Hodgkin's lymphoma, liver cancer, gastric cancer, esophageal cancer, colorectal cancer, cervical cancer, breast cancer, leukemia, lymphoma, neuroblastoma, glioblastoma, non-small cell lung cancer, head and neck squamous cell carcinoma, small cell lung cancer, melanoma, myeloma, ovarian cancer, pancreatic cancer, endometrial cancer, prostate cancer, urothelial cancer, thyroid cancer, and testicular cancer.

Examples of a chemotherapeutic agent include, but are not limited to, amsacrine, asparaginase, azathioprine, bisantrene, bleomycin, busulfan, capecitabine, carboplatin, carmustine, chlorambucil, cisplatin, cladribine, clofarabine, cyclophosphamide, cytarabine, dacarbazine, dactinomycin, daunorubicin, docetaxel, doxorubicin, epirubicin, etoposide, flavopiridol, fludarabine, fluorouracil, gemcitabine, idarubicin, ifosfamide, irinotecan, hydroxyurea, leucovorin, liposomal daunorubicin, liposomal doxorubicin, lomustine, mechlorethamine, melphalan, mercaptopurine, mesna, methotrexate, mitomycin, mitoxantrone, oxaliplatin, paclitaxel, pemetrexed, pentostatin, procarbazine, satraplatin, streptozocin, tegafur-uracil, temozolomide, teniposide, thioguanine, thiotepa, treosulfan, topotecan, vinblastine, vincristine, vindesine and vinorelbine.

Embodiments include compositions and methods for imaging cells expressing ABCG2 by administering an ABCG2 inhibitor identified by the above method, wherein the ABCG2 inhibitors are labeled with one or more radioisotopes. In exemplary embodiments, the cells are stem cells or cancer stem cells.

Embodiment include compositions and methods for the treatment of a central nervous system disorder by administering to a patient suffering therefrom an inhibitor of ABCG2 identified by the above method, wherein the ABCG2 inhibitor facilitates drug delivery across the blood-brain barrier. Examples of a central nervous system disorder include, but are not limited to, schizophrenia, Alzheimer's disease, Parkinson's disease, Huntington's disease, bipolar disorder, multiple sclerosis, dementia, stroke, and depression.

Other embodiments include methods for improving the oral absorption of a pharmaceutically active agent by administering an ABCG2 inhibitor identified by the above method in combination with an orally active pharmaceutically active agent. In such embodiments, the oral absorption of the pharmaceutically active agent is greater in combination with the ABCG2 inhibitor than the oral absorption of the pharmaceutically active agent without the ABCG2 inhibitor.

Other embodiments include methods for increasing transport of a CNS active agent across the blood-brain barrier by administering an ABCG2 inhibitor identified by the above method in combination with a CNS active agent. In such embodiments, the transport of the CNS active agent across the blood-brain barrier is greater in combination with the ABCG2 inhibitor than the transport of the CNS active agent without the ABCG2 inhibitor.

Other embodiments include methods of improving the effectiveness of a chemotherapeutic agent by administering a chemotherapeutic agent in combination with an ABCG2 inhibitor identified by the above method. In such embodiments, effectiveness of the chemotherapeutic agent is greater in combination with the ABCG2 inhibitor than the effectiveness of the chemotherapeutic agent without the ABCG2 inhibitor.

Other embodiments include methods of treating multiple drug resistant cancer by administering a chemotherapeutic agent in combination with an ABCG2 inhibitor identified by the above method. In such embodiments, an otherwise resistant cancer becomes more sensitive to the chemotherapeutic agent when administered in combination with the ABCG2 inhibitor. In the above embodiments, the ABCG2 inhibitor may further be selected from the compounds listed in tables 2-6. In other embodiments, the ABCG2 inhibitor may be selected from the compounds listed in table 2, but excluding compounds in tables 3 or 5. In other embodiments, the ABCG2 inhibitor may be selected from the compounds shown in table 4. In some embodiments, the ABCG2 inhibitor is selected from glafenine, tracazolate, calcimycin (A23187), doxazosin verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil emodin, flubendazole, metyrapone (2-methyl-1,2-dipyridin-3-yl-propan-l-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof. In other embodiments, the ABCG2 inhibitor may be selected from doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof. In some embodiments, the ABCG2 inhibitor may be Doxazosin, flavoxate, dihydroergotamine or combinations thereof.

Other features of the invention will be apparent from the detailed description, and from the claims.

### BRIEF DESCRIPTION OF THE DRAWINGS

FIG. 1. Mitoxantrone (MTX) resensitization assay. NCI-H460/MX20 cells were treated for three days with or without MTX (30 μM in panel A, and 15 μM in panels B, C and D), in the presence of a potential inhibitor (20 μM in panel A, B and C, 1 μM in panel D), and surviving cells were assessed with the XTT assay. Survival rates were expressed as percentages normalized by the data obtained in the negative control where no MTX or any compound was added. 10 μM FTC was used as a positive control. Numbers on top of bar pairs are survival rates caused by each compound normalized by its cytotoxicity. Data are presented as mean±SEM, n=3.

FIG. 2. Effect of selected positive hits on ABCG2 function shown by flow cytometry analysis of the BODIPY-prazosin dye uptake assay. HEK293/ABCG2 cells were incubated in BODIPY-prazosin in the absence (open curve) or presence of a compound (20 μM, filled curve) as described in the Materials and Methods. FTC (10 μM) was used as a positive control.

FIG. 3. ABCG2 inhibitors cause a dose-dependent increase of bioluminescence signal in HEK293/ABCG2 cells expressing fLuc. Cells were imaged in medium containing 50 μg/mL D-luciferin and increasing concentrations of glafenine (A), doxazosin mesylate (B), flavoxate hydrochloride (C), clebopride maleate (D), and FTC (E), and bioluminescence signal was quantified. The data were plotted and the IC₅₀ value of each ABCG2 inhibitor was calculated with GraphPad Prism version 4.0 for Windows (GraphPad Software, San Diego, Calif.) using variable-slope logistic nonlinear regression analysis. Mean±SEM, n=3.

FIG. 4. ABCG2 inhibitors resensitize ABCG2-overexpressing HEK293 cells to MTX treatment. Cells were plated at a density of 1×10⁴ cells per well in 96-well plates, and allowed to attach before incubated in medium containing an ABCG2 inhibitor and/or MTX for 3 days. Cell viabilities were assessed with the XTT assay and expressed as percentages of the control that was treated with MTX alone. Mean±SEM, n=3.

FIG. 5. ABCG2 inhibitors also resensitize Pgp (A) or MRP1 (B) overexpressing MDCKII cells to colchicine treatment. Cells were plated at a density of 1×10⁴ cells per well in 96-well plate, and allowed to attach before incubated in medium containing an ABCG2 inhibitor and/or colchicine for 2 days. Cell viabilities were assessed with the XTT assay and expressed as percentages of the control which was treated with colchicine alone. Mean±SEM, n=3.

FIG. 6. Glafenine inhibits ABCG2 activity in a living mouse as shown by BLI. HEK293/empty/fLuc (control) and HEK293/ABCG2/fLuc cells were implanted to the flanks (left and right, respectively) of immunocompromised (nude) mice. A) A representative mouse showing BLI acquired 30 min after administration of D-luciferin i.p., immediately before administration of glafenine (25 mg/kg). B) The same mouse as in (A) imaged 15 min after i.v. glafenine administration. C) Time course of BLI signal from both control and ABCG2 overexpressing xenografts before and after glafenine injection. The BLI signal from ABCG2 transfected xenografts increased up to ˜11.6- and ˜17.4-fold (right front and rear, respectively), while the BLI signal from the control xenograft increased only ˜2.6-fold, compared to their signals immediately before glafenine injection. The arrow indicates the time of glafenine injection.

### DETAILED DESCRIPTION

### Definitions

As used herein, “agent” is a non-peptide, small molecule compound.

By “analog” is meant an agent having structural or functional homology to a reference agent.

By “cell substrate” is meant the cellular or acellular material (e.g., extracellular matrix, polypeptides, peptides, or other molecular components) that is in contact with the cell.

By “control” is meant a standard or reference condition.

By “disease” is meant any condition or disorder that damages or interferes with the normal function of a cell, tissue, organ or subject.

By “effective amount” is meant the amount of an agent required to ameliorate the symptoms of a disease relative to an untreated subject. The effective amount of an active therapeutic agent for the treatment of a disease or injury varies depending upon the manner of administration, the age, body weight, and general health of the subject. Ultimately, the attending clinician will decide the appropriate amount and dosage regimen.

By “modifies” is meant alters. An agent that modifies a cell, substrate, or cellular environment produces a biochemical alteration in a component (e.g., polypeptide, nucleotide, or molecular component) of the cell, substrate, or cellular environment.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, a “prodrug” is a pharmacologically inactive compound that is converted into a pharmacologically active agent by a metabolic transformation.

By “subject” is meant a mammal, including, but not limited to, a human or non-human mammal, such as a bovine, equine, canine, ovine, or feline.

By “therapeutic delivery device” is meant any device that provides for the release of a therapeutic agent. Exemplary therapeutic delivery devices include tablets and pills, described below, as well as syringes, osmotic pumps, indwelling catheters, delayed-release and sustained-release biomaterials.

As used herein, the terms “treat,” treating,” “treatment,” “therapeutic” and the like refer to reducing or ameliorating a disorder and/or symptoms associated therewith. It will be appreciated that, although not precluded, treating a disorder or condition does not require that the disorder, condition or symptoms associated therewith be completely eliminated.

By “variant” is meant an agent having structural homology to a reference agent/compound but varying from the reference in its biological activity.

As used herein, the term “ABCG2 inhibitor” defines a compound that reduces or decreases the activity of the ABCG2 transporter protein, resulting in a decrease in transport of an ABCG2 substrate.

As used herein, the term “ABCG2 substrate” is a compound that is a substrate of ABCG2 and is transported by the protein (i.e. ABCG2). ABCG2 substrates are described, for example, by Robey et al. (Cancer Metathesis Reviews, vol. 26, pp. 39-57, 2007), which is incorporated by reference in its entirety. Examples of ABCG2 substrates include Mitoxantrone, Daunorubicin, Doxorubicin, Epirubicin, Bisantrene, Flaopiridol, Etoposide, Teniposide, 9-aminocamptothecin, topotecan, irinotecan, SN-38, diflomotecan, homocamptothecin, DX-8951f, BNP1350, J-107088, NB-506, UCN-01, methotrexate, methotrexate di-glutamate, methotrexate tri-glutamate, GW1843, Tomudex, Imatinib, Gefitinib, CI1033, Pheophorbide a, Pyropheophrobide a methyl ester, chlorine e6, protoporphyrin IX. Other ABCG2 substrates include statins such as rosuvastatin, pitavastatin, pravastatin, and cervastatin; flavonoids, such as genestein and quercetin; antibiotics, such as nitrofurantoin, fluoroquinolones; and antihelminthic benzimidazoles.

### Assay

Emobidments of the invention include methods of identifying ABCG2 inhibitors. These method include imaging the bioluminescence of cells that express ABCG2 and firefly luciferase (fLuc). The cultures further include D-luciferin, and one or more test compound(s). ABCG2 inhibitors are identified as those compounds that produce an increase of at least 2 fold in bioluminescence, for example an increase of at least 5 fold in bioluminescence.

The culture may be prepared by adding a test compound to produce a predetermined concentration. The concentration may be any suitable concentration that does not otherwise interfere with the performance of the assay. For instance, the test compound should not be present in a high concentrations that may be cyctotoxic and kill the cells in the assay. In some embodiments the concentration of test compound in the culture may be between about 0.1 nM and about 100 μM. The addition of a test compound produces a transformation within the cell, and may inhibit ABCG2 or interact with other proteins or enzymes in the cell.

D-luciferin is also added to produce a predetermined concentration. The concentration of D-luciferin may be any concentration sufficient to produce a detectable bioluminescence signal. The concentration of D-luciferin in the culture may be adjusted as desired, for instance to optimize the signal to background ratio, or to prevent saturation of the detector(s). In some embodiments, the concentration of D-luciferin in the culture may be between about 1 μg/mL and about 1000 μg/mL, between about 1 μg/mL and about 500 μg/mL, or between about 1 μg/mL and about 100 μg/mL. In some embodiments, the concentration of D-luciferin may be about 50 μg/mL. D-luciferin is transformed by firefly luciferase (fLuc) in the culture to produce bioluminescence, which is detected by imaging the culture.

Any cell line may be used which expresses both ABGC2 and fLuc. In some embodiments, the cell line may adhere strongly to a substrate, such as a multi-well plate. Such cell lines may be suitable for use for high-throughput screening. In general, any cell line may be used where ABCG2 is overexpressed, and which may be transformed or transfected to express fLuc. In some embodiments, the cell line may be NCI-H460/MX20. In some embodiments, the cell line may be HEK293 cells.

A negative control is prepared identical to the test assay, where no test compound is added. In some embodiments, an amount of solvent having no test compound is added in the same quantity used to add the test compound. The negative control produces a certain level of bioluminescence based on the amount of ABCG2 expression in the cell, the concentration of D-luciferin in the culture, and the amount of time after addition of D-luciferin. The bioluminescence of the negative control determines the amount bioluminescence needed to identify an ABCG2 inhibitor. A 2-fold or greater increase in bioluminescence, compared with the negative control, is sufficient to identify an ABCG2 inhibitor using this method. In some embodiments, the threshold may be higher, for instance 3-fold or greater, 4-fold or greater, 5-fold or greater, 6-fold or greater, 8-fold or greater, 10-fold or greater, 15-fold or greater or 20-fold or greater increase may be used to select ABCG2 inhibitors using this method.

The bioluminescence may be measured at any time after the culture is prepared (i.e. after the test compound and D-luciferin have been added), so long as detectable amounts of bioluminescence are produced. In some embodiments, the bioluminescence is measured at multiple time points after addition of the last component. The bioluminescence may then be graphed against time to determine an optimal time of measurement. For example, the optimal time for measurement may be the time point that gives maximum signal. The optimal time may be a time point selected for a different reason, such as the amount of time needed to move the culture into a detector.

Bioluminescence may be detected by any suitable means. In some embodiments, an imaging device is used to quantify the amount of bioluminescence in different cultures. Examples of imaging devices include luminometers and plate readers. Examples of imaging devices include, Xenogen IVIS imaging machine, or other imaging device that can image bioluminescence such as Kodak imager. When identification is conducted in multi-well plates, light output from each well may be quantified at the desired time point, and the signal-to-background (S/B) ratio of the light output from each test compound divided by that from the negative control calculated. This S/B ratio indicates the potency of ABCG2 inhibition.

In some embodiments, the assay may be performed at different concentrations of test compound. The results of these assays may be used to calculate IC₅₀ or EC₅₀ values for the test compounds.

The activity of the ABCG2 inhibitors identified by the methods described may be confirmed by secondary assays. In some embodiments, the activity of the ABCG2 inhibitors may be confirmed by a resensitization assay or a dye uptake assay.

A resensitization assay involves treating cells (usually cancer cells) that express ABCG2 with a chemotherapeutic agent at a concentration that would not normally kill the cells (due to resistance based on ABCG2 expression), in combination with an inhibitor identified by the described assay at a concentration sufficient to inhibit ABCG2. Suitable chemotherapeutic agents are those which are substrates for ABCG2. In some embodiments, the chemotherapeutic agent is Mitoxantrone (MTX). Other suitable chemotherapeutic agents include daunorubicin, doxorubicin, bisantrene, topotecan and flavopiridol. In the resensitization assay, the ABCG2 inhibitor causes the resistant cells to become more sensitive to the chemotherapeutic agent. Without being bound by theory, a possible mechanism by which sensitivity is increased functions by reducing or eliminating efflux of the chemotherapeutic agent from the cell by ABCG2. Hence, the cells become “resensitized” to the chemotherapeutic agent, and confirm the activity of the ABCG2 inhibitor.

A dye uptake assay involves treating cells that express ABCG2 with a dye and an ABCG2 inhibitor. Suitable dyes are those which are substrates for ABCG2. In some embodiments, the dye is BODIPY-prazosin. In a dye uptake confirmation assay, the dye and ABCG2 inhibitor enter the cells, where the ABCG2 inhibitor reduces ABCG2-mediated efflux of the dye out of the cells. As a result, a greater concentration of dye is present in cells treated with ABCG2 inhibitor, compared with untreated cells. The increase in dye may result in increased color, UV absorbance or fluorescence, which can be measured. An increase in color, UV absorbance or fluorescence indicates successful inhibition of ABCG2, and confirming the activity of the ABCG2 inhibitor.

Other embodiments include compositions of an ABCG2 inhibitor identified by the described method, a second pharmaceutically active agent, and a pharmaceutically acceptable carrier or excipient.

In some embodiments, the pharmaceutically active agent may be an orally available pharmaceutically active agent. In some embodiments, the orally available agent may be absorbed, at least in part, in the small intestine. In some embodiments, the orally available pharmaceutically active agent is an ABCG2 substrate. The pharmaceutically active agent is present in a pharmaceutically active amount in the composition, and the ABCG2 inhibitor is present in an amount sufficient to improve the oral bioavailability of the pharmaceutically active agent. In these embodiments, it is believed that the ABCG2 inhibitor prevents ABCG2-mediated efflux of the pharmaceutically active agent from the epithelium of the small intestine, resulting in an increase in oral absorption. In other words, the oral absorption of the pharmaceutically active agent in combination with the ABCG2 inhibitor is greater than the oral absorption of the pharmaceutically active agent in the absence of the ABCG2 inhibitor. Other mechanisms may also be involved

In other embodiments, the pharmaceutically active agent may be a chemotherapeutic agent. In some embodiments, the chemotherapeutic agent may be an ABCG2 substrate. The chemotherapeutic agent is present in a therapeutically effective amount. The ABCG2 inhibitor is present in an amount sufficient to increase the effectiveness of the chemotherapeutic agent. In these embodiments, the ABCG2 inhibitor may reduce ABCG2-mediated efflux of the chemotherapeutic agent from the cancer cell, resulting in an increase in effectiveness of the chemotherapeutic agent. In other words, the therapeutic effectiveness of the chemotherapeutic agent in combination with the ABCG2 inhibitor is greater than the therapeutic effectiveness of the chemotherapeutic agent in the absence of the ABCG2 inhibitor. Other mechanisms may also account for increased effectiveness.

In other embodiments, the pharmaceutically active agent is a CNS active agent. A “CNS active agent” is a therapeutic agent active in the central nervous system. For example, the therapeutic agent may be used for treatment of a central nervous system disorder, or may be a therapeutic agent used for treatment of a disease such as viral or bacterial infections, or cancer in the central nervous system. In some embodiments, the CNS active agent is an ABCG2 substrate. The CNS active agent is present in a therapeutically effective amount, and the ABCG2 inhibitor is present in an amount sufficient to increase the transport of the therapeutic agent across the blood-brain barrier. In these embodiments, the ABCG2 inhibitor may reduce ABCG2-mediated efflux of the CNS active agent across the blood-brain barrier, and out of the central nervous system. In other words, the therapeutic effectiveness of the CNS active agent in combination with the ABCG2 inhibitor is greater than the therapeutic effectiveness of the CNS active agent in the absence of the ABCG2 inhibitor. Likewise, the concentration of CNS active agent in the central nervous system, in combination with the ABCG2 inhibitor is greater than the concentration of CNS active agent in the central nervous system in the absence of the ABCG2 inhibitor. Other mechanisms may also account for this increase.

Other embodiments include methods for increasing the oral absorption of a pharmaceutically active agent by administering an ABCG2 inhibitor identified by the above method in combination with an orally active pharmaceutically active agent. In such embodiments, the oral absorption of the pharmaceutically active agent is greater in combination with the ABCG2 inhibitor than the oral absorption of the pharmaceutically active agent without the ABCG2 inhibitor. In some embodiments, the orally available agent may be absorbed, at least in part, in the small intestine. In some embodiments, the orally available pharmaceutically active agent is an ABCG2 substrate.

Other embodiments include methods of improving the effectiveness of a chemotherapeutic agent by administering a chemotherapeutic agent in combination with an ABCG2 inhibitor identified by the above method. In such embodiments, effectiveness of the chemotherapeutic agent is greater in combination with the ABCG2 inhibitor than the effectiveness of the chemotherapeutic agent without the ABCG2 inhibitor. In some embodiments, the chemotherapeutic agent may be an ABCG2 substrate.

Other embodiments include methods for increasing transport of a CNS active agent across the blood-brain barrier by administering an ABCG2 inhibitor identified by the above method in combination with a CNS active agent. Examples of central nervous system disorders include schizophrenia, Alzheimer's disease, Parkinson's disease, Huntington's disease, bipolar disorder, multiple sclerosis, dementia, stroke, and depression. In some exemplary embodiments, the central nervous system disorder may be schizophrenia, Alzheimer's disease, Parkinson's disease, or Huntington's disease. Examples of CNS active agents also include chemotherapeutic agents used to treat cancers in the CNS. In such embodiments, the transport of the CNS active agent across the blood-brain barrier is greater in combination with the ABCG2 inhibitor than the transport of the CNS active agent without the ABCG2 inhibitor. In some embodiments, the CNS active agent is an ABCG2 substrate.

In all the above embodiments, the ABCG2 inhibitor may further be selected from the compounds shown in tables 2-6. In some exemplary embodiments, the ABCG2 inhibitor may be selected from the compounds present in table 2, but not in tables 3 or 5. In other exemplary embodiments, the ABCG2 inhibitor may be selected from the compounds shown in table 4. In exemplary embodiments, the ABCG2 inhibitor is selected from glafenine, tracazolate, calcimycin (A23187), doxazosin verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof In other exemplary embodiments, the ABCG2 inhibitor may be selected from doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof In some exemplary embodiments, the ABCG2 inhibitor may be Doxazosin, flavoxate, dihydroergotamine or combinations thereof.

Embodiments of the invention include methods of treating a cellular proliferative disorder by administering to a patient in need of treatment an ABCG2 inhibitor identified by the methods described herein. Other embodiments include methods wherein the ABCG2 inhibitor is administered in combination with an additional chemotherapeutic agent. Examples of cellular proliferative disorders include, but are not limited to, acute myelogenous leukemia, acute lymphoblastic leukemia, multiple myeloma, non-Hodgkin's lymphoma, Hodgkin's lymphoma, liver cancer, gastric cancer, esophageal cancer, colorectal cancer, cervical cancer, breast cancer, leukemia, lymphoma, neuroblastoma, glioblastoma, non-small cell lung cancer, head and neck squamous cell carcinoma, small cell lung cancer, melanoma, myeloma, ovarian cancer, pancreatic cancer, endometrial cancer, prostate cancer, urothelial cancer, thyroid cancer, and testicular cancer. In exemplary embodiments, the cellular proliferative disorder is acute myeloid leukemia (AML), acute myelogenous leukemia (AML) and acute lymphoblastic leukemia (ALL), and tumors from the digestive tract, endometrium, lung and melanoma. Examples of the additional chemotherapeutic agent include amsacrine, asparaginase, azathioprine, bisantrene, bleomycin, busulfan, capecitabine, carboplatin, carmustine, chlorambucil, cisplatin, cladribine, clofarabine, cyclophosphamide, cytarabine, dacarbazine, dactinomycin, daunorubicin, docetaxel, doxorubicin, epirubicin, etoposide, flavopiridol, fludarabine, fluorouracil, gemcitabine, idarubicin, ifosfamide, irinotecan, hydroxyurea, leucovorin, liposomal daunorubicin, liposomal doxorubicin, lomustine, mechlorethamine, melphalan, mercaptopurine, mesna, methotrexate, mitomycin, mitoxantrone, oxaliplatin, paclitaxel, pemetrexed, pentostatin, procarbazine, satraplatin, streptozocin, tegafur-uracil, temozolomide, teniposide, thioguanine, thiotepa, treosulfan, topotecan, vinblastine, vincristine, vindesine and vinorelbine. In some embodiments, multiple additional chemotherapeutic agents may be used. In some exemplary embodiments, the ABCG2 inhibitor is selected from the compounds described in tables 2-6. In some exemplary embodiments, the ABCG2 inhibitor may be selected from the compounds present in table 2, but not in tables 3 or 5. In other exemplary embodiments, the ABCG2 inhibitor may be selected from the compounds shown in table 4. In exemplary embodiments, the ABCG2 inhibitor is selected from glafenine, tracazolate, calcimycin (A23187), doxazosin verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof In other exemplary embodiments, the ABCG2 inhibitor may be selected from doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof. In other exemplary embodiments, the ABCG2 inhibitor may be Doxazosin, flavoxate, dihydroergotamine or combinations thereof.

Embodiments include methods of treating multiple drug resistant cancers by administering an ABCG2 inhibitor identified by the methods described and a therapeutically effective amount of a chemotherapeutic agent different from the ABCG2 inhibitor. In some exemplary embodiments, the ABCG2 inhibitor is selected from the compounds described in tables 2-6. In some exemplary embodiments, the ABCG2 inhibitor may be selected from the compounds present in table 2, but not in tables 3 or 5. In other exemplary embodiments, the ABCG2 inhibitor may be selected from the compounds shown in table 4. In some exemplary embodiments, the ABCG2 inhibitor is selected from glafenine, tracazolate, calcimycin (A23187), doxazosin verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof In other exemplary embodiments, the ABCG2 inhibitor may be selected from doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof In some exemplary embodiments, the ABCG2 inhibitor may be Doxazosin, flavoxate, dihydroergotamine or combinations thereof. In some exemplary embodiments, the chemotherapeutic agent may be amsacrine, asparaginase, azathioprine, bisantrene, bleomycin, busulfan, capecitabine, carboplatin, carmustine, chlorambucil, cisplatin, cladribine, clofarabine, cyclophosphamide, cytarabine, dacarbazine, dactinomycin, daunorubicin, docetaxel, doxorubicin, epirubicin, etoposide, flavopiridol, fludarabine, fluorouracil, gemcitabine, idarubicin, ifosfamide, irinotecan, hydroxyurea, leucovorin, liposomal daunorubicin, liposomal doxorubicin, lomustine, mechlorethamine, melphalan, mercaptopurine, mesna, methotrexate, mitomycin, mitoxantrone, oxaliplatin, paclitaxel, pemetrexed, pentostatin, procarbazine, satraplatin, streptozocin, tegafur-uracil, temozolomide, teniposide, thioguanine, thiotepa, treosulfan, topotecan, vinblastine, vincristine, vindesine, vinorelbine or combinations thereof.

In other exemplary embodiments, the ABCG2 inhibitor may also inhibit other ATP-binding cassette (ABC) transporters, such as P-glycoprotein (Pgp) or multiple drug resistance protein 1 (MGP1). Examples of compounds which inhibit ABCG2 in addition to another ABC transporter (e.g. Pgp and/or MGP1) include cyclosporine, Doxazosin, Rotenone and Glafenine. Inhibitory activity against other ABC transporters may be determined using assays known in the art.

Embodiments include methods of treating a central nervous system disorder by administering to a patient in need of treatment an ABCG2 inhibitor identified by the methods described above and a therapeutically effective amount of a CNS active agent. Examples of central nervous system disorders include schizophrenia, Alzheimer's disease, Parkinson's disease, Huntington's disease, bipolar disorder, multiple sclerosis, dementia, stroke, and depression. In some exemplary embodiments, the central nervous system disorder may be schizophrenia, Alzheimer's disease, Parkinson's disease, or Huntington's disease. Examples of CNS active agents also include chemotherapeutic agents used to treat cancers in the CNS. In some exemplary embodiments, the ABCG2 inhibitor is selected from the compounds described in tables 2-6. In some exemplary embodiments, the ABCG2 inhibitor may be selected from the compounds present in table 2, but not in tables 3 or 5. In other exemplary embodiments, the ABCG2 inhibitor may be selected from the compounds shown in table 4. In some exemplary embodiments, the ABCG2 inhibitor is selected from glafenine, tracazolate, calcimycin (A23187), doxazosin verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof In other exemplary embodiments, the ABCG2 inhibitor may be selected from doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof. In some exemplary embodiments, the ABCG2 inhibitor may be Doxazosin, flavoxate, dihydroergotamine or combinations thereof.

Embodiments include methods of imaging cells, tumors, tissues, or organs that express ABCG2 by administering an effective amount of an ABCG2 inhibitor identified by the methods described above, that has been labeled with one or more radioisotopes or derivatized with one or more fluorescent dyes. For example, the ABCG2 inhibitor may be radiolabeled with an imaging radionuclide such as ¹²³I, ¹²⁴I, ¹²⁵I, ⁶⁸Ga, ¹⁸F, ¹¹C, ^{99m}Tc, ¹¹¹In or derivatized with an optical moiety such as FITC, marina blue, a carbocyanine dye, etc. Such isotope labeled and derivatized compounds are known in the art or may be prepared according to known processes.

In some exemplary embodiments, the ABCG2 inhibitor is selected from the compounds described in tables 2-6. In some exemplary embodiments, the ABCG2 inhibitor may be selected from the compounds present in table 2, but not in tables 3 or 5. In other exemplary embodiments, the ABCG2 inhibitor may be selected from the compounds shown in table 4. In some exemplary embodiments, the ABCG2 inhibitor is selected from glafenine, tracazolate, calcimycin (A23 187), doxazosin verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, and oligomycin. In some exemplary embodiments, the ABCG2 inhibitor may be Doxazosin, flavoxate, or dihydroergotamine.

A cell-based, high-throughput assay to uncover new inhibitors of ABCG2 has been developed. This assay builds upon the discovery that D-luciferin, the substrate of fLuc, is a specific substrate of ABCG2. The assay uses Bioluminescence Imaging (BLI) to screen for ABCG2 inhibitors (Zhang et al, “ABCG2/BCRP expression modulates D-Luciferin based bioluminescence imaging,” Cancer research, vol. 67, no. 19, pp. 9389-9397, 2007). The screening of 3,273 compounds identified 219 candidate ABCG2 inhibitors with at least a two-fold signal enhancement over controls, ˜60% of which have been previously reported as ABCG2 inhibitors, including gefitinib, prazosin, and harmine. The ability to identify known ABC transporter inhibitors, both potent and weak, demonstrates that the assay is sensitive and reliable. The results also demonstrate the ability of the assay to identify previously unknown ABCG2 inhibitors. Approximately 40% of the 219 potent and about 84% of the approximately 150 less potent compounds have never been reported previously as being inhibitors or substrates of an ABC transporter. The less potent compounds, in particular, may be difficult to identify with other methods.

The benefits of the present BLI assay was further demonstrated by confirming the ABCG2 inhibitory activity of almost all of the novel ABCG2 inhibitors uncovered, indicating a low false-positive rate. A screen of more than 70,000 compounds by an assay using pure fLuc found no activator of the luciferase-coupled reaction that could enhance the luminescent signal (Auld, D. S., et al., Characterization of chemical libraries for luciferase inhibitory activity. J Med Chem, 2008. 51(8): p. 2372-86). This may account for the low false positive rate. Signal enhancement seen in the BLI assay is attributed only to the increased intracellular concentration of D-luciferin upon administration of putative ABCG2 inhibitors from the screening library.

Twenty eight candidate ABCG2 inhibitors with over five-fold signal enhancement were subjected to a MTX resensitization assay, and 16 of them were also tested with a BODIPY-prazosin dye uptake assay. Except for seven compounds that were too cytotoxic to be tested, all were confirmed by the MTX resensitization assay.

The BLI-based assay is very sensitive with no false negatives uncovered. While the results of the MTX resensitization assay were as expected, those of the BODIPY-prazosin dye uptake assay were intriguing. Only nine out of 16 compounds tested were confirmed by this fluorescence-based assay and seven (˜44%) failed this assay altogether. Five of the seven compounds were confirmed by the MTX resensitization assay, and two were too cytotoxic to test (Table 6). Notably, MTX resistance is the hallmark of the ABCG2 phenotype (Doyle et al., “Multidrug resistance mediated by the breast cancer resistance protein BCRP (ABCG2),” Oncogene, vol. 22, no. 47, pp. 7340-7358, 2003; Bates et al., “The role of half-transporters in multidrug resistance,” Journal of bioenergetics and biomembranes, vol. 33, no. 6, pp. 503-511, 2001), therefore, this discrepancy suggests a high false negative rate for the BODIPY-prazosin assay.

To understand the discrepancy better, the structures of the seven compounds that could not be confirmed by the BODIPY-prazosin assay were analyzed. Metyraphone, acepromazine, peperacetazine and acetophenazine have an aromatic ketone functional group, which can act as an electron acceptor and deactivate the singlet state of BODIPY via an intermolecular electron-transfer process (Perez-Prieto et al., “Aromatic ketones as photocatalysts: combined action as triplet photosensitiser and ground state electron acceptor,” Chemphyschem, vol. 7, no. 10, pp. 2077-2080, 2006; Matsumoto et al., “A thiol-reactive fluorescence probe based on donor-excited photoinduced electron transfer: key role of ortho substitution,” Organic letters, vol. 9, no. 17, pp. 3375-3377, 2007). Porphyrin in verteporin and benzopteridine in riboflavin can quench the fluorescence of BODIPY by way of photoinduced electron transfer (Ulrich et al., “The chemistry of fluorescent bodipy dyes: versatility unsurpassed,” Angewandte Chemie (International ed.), vol. 47, no. 7, pp. 1184-1201, 2008; Koenig et al., “Photoinduced Electron Transfer in a Phenothiazine-Riboflavin Dyad Assembled by Zinc-Imide Coordination in Water,” Journal of the American Chemical Society, vol. 121, no. 8, p. 7, 1999). It has been reported previously that the fluorescence of several dyes used to probe mitochondrial transmembrane potential can be quenched by some anticancer drugs, including adaphostin, MTX and amsacrine (Le et al., “Adaphostin and other anticancer drugs quench the fluorescence of mitochondrial potential probes,” Cell death and differentiation, vol. 13, no. 1, pp. 151-159, 2006). Accordingly, fluorescence-based assays must be cautiously applied. The implication of this finding is significant. Since fluorescence-based assays have seen the most use in discovering new ABCG2 inhibitors (Robey, et al, “Mutations at amino-acid 482 in the ABCG2 gene affect substrate and antagonist specificity,” British journal of cancer, vol. 89, no. 10, pp. 1971-1978, 2003, Rajagopal et al., Subcellular localization and activity of multidrug resistance proteins,” Molecular biology of the cell, vol. 14, no. 8, pp. 3389-3399, 2003; Mogi et al., “Akt signaling regulates side population cell phenotype via Bcrpl translocation,” The Journal of biological chemistry, vol. 278, no. 40, pp. 39068-39075, 2003; Henrich et al., A high-throughput cell-based assay for inhibitors of ABCG2 activity,” J Biomol Screen, vol. 11, no. 2, pp. 176-183, 2006), it is possible that many ABCG2 inhibitors that quench fluorescence have been missed. The BLI-based screening assay described here has the advantage of not being prone to such an artifact. That advantage was demonstrated by a search of the Johns Hopkins Clinical Compound Library (JHCCL) for previously known ABCG2 inhibitors, which revealed that the BLI-based assay missed none of them.

The BLI-based assay is efficient, compared with other assays, due to the elimination of incubation and wash steps. Several hundred drugs can be screened in one day using the BLI assay as described herein, with many thousands of drugs possible if the technique is automated. False negatives caused by cytotoxicity in extended incubation are not a concern. While pore-forming proteins or detergents that disrupt cell membranes may cause false positives because of the leakage of D-luciferin into cells, no such reagents were identified in screen using the method.

Candidate ABCG2 inhibitors obtained from a screen of the JHCCL are categorized based on their therapeutic effects, and can be clustered into several classes, including drugs affecting cardiovascular and central nervous system (CNS), and digestive systems, among others (Table 1).

TABLE 1
Categories of potential ABCG2 inhibitors

| | Posi- | Repviously | Not Previously |
|---|---|---|---|
| Categories | tive | Reported | Reported |
| CNS (antiparkinsonian, | 31 | Y (20) | 11 |
| antipsychotic, etc.) | | | |
| glucocorticoid, antiinflammatory | 7 | Y(2) | 5 |
| cathartic, laxative, dirurectic | 4 | Y(3) | 1 |
| cardiovascular | 15 | Y(8) | 7 |
| migraine, antianginal/pain related | 7 | Y(5) | 2 |
| estrogen related | 7 | Y(5) | 2 |
| antihistamine | 8 | Y(6) | 2 |
| antibiotic | 25 | Y(11) | 14 |
| oil, tar (therapeutic plant) | 11 | 0 | 11 |
| antiemetic | 12 | Y(7) | 5 |
| antiviral | 9 | Y(8) | 1 |
| antispasmodic, muscle relaxant | 6 | Y(1) | 5 |
| anthelmintic | 7 | Y(5) | 2 |

### Compounds

In all embodiments, the ABCG2 inhibitor or other active compounds may be present as pharmaceutically acceptable salts or other derivatives, such as ether derivatives, ester derivatives, acid derivatives, and aqueous solubility altering derivatives of the active compound. Derivatives include all individual enantiomers, diastereomers, racemates, and other isomers of the compounds. Derivatives also include all polymorphs and solvates, such as hydrates and those formed with organic solvents, of the compounds. Such isomers, polymorphs, and solvates may be prepared by methods known in the art, such as by regiospecific and/or enantioselective synthesis and resolution.

The ability to prepare salts depends on the acidity of basicity of the compounds. Suitable salts of the compounds include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds.

### Pharmaceutical Compositions

In some embodiments the compositions may include one or more than one ABCG2 inhibitor, one or more other pharmaceutically active agent, and may further contain other suitable substances and excipients, including but not limited to physiologically acceptable buffering agents, stabilizers (e.g. antioxidants), flavoring agents, agents to effect the solubilization of the compound, and the like.

In other embodiments, the composition may be in any suitable form such as a solution, a suspension, an emulsion, an infusion device, or a delivery device for implantation or it may be presented as a dry powder to be reconstituted with water or another suitable vehicle before use. The composition may include suitable parenterally acceptable carriers and/or excipients.

In other embodiments, the compositions may comprise an effective amount of an inhibitor and/or other pharmaceutically active agent in a physiologically-acceptable carrier. The carrier may take a wide variety of forms depending on the form of preparation desired for a particular route of administration. Suitable carriers and their formulation are described, for example, in Remington's Pharmaceutical Sciences by E. W. Martin.

In some embodiments, the inhibitor may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) or oral administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

In some embodiments, the compositions may be in a form suitable for administration by sterile injection. In one example, to prepare such a composition, the compositions(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). For parenteral formulations, the carrier will usually comprise sterile water, though other ingredients, for example, ingredients that aid solubility or for preservation, may be included. Injectable solutions may also be prepared in which case appropriate stabilizing agents may be employed.

Formulations suitable for parenteral administration usually comprise a sterile aqueous preparation of the inhibitor, which may be isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery or localized delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

In some embodiments, the compositions may be in a form suitable for oral administration. In compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. If desired, tablets may be sugar coated or enteric coated by standard techniques.

Compositions suitable for oral administration may be presented as discrete units such as capsules, cachets, tablets, or lozenges, each containing a predetermined amount of the active ingredient as a powder or granules. Optionally, a suspension in an aqueous liquor or a non-aqueous liquid may be employed, such as a syrup, an elixir, an emulsion, or a draught. Formulations for oral use include tablets containing active ingredient(s) in a mixture with pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

A syrup may be made by adding the inhibitor to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

In some embodiments, the composition may be in a form of nasal or other mucosal spray formulations (e.g. inhalable forms). These formulations can include purified aqueous solutions of the active compounds with preservative agents and isotonic agents. Such formulations can be adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

In some embodiments, the composition may be in a form suitable for rectal administration. These formulations may be presented as a suppository with a suitable carrier such as cocoa butter, hydrogenated fats, or hydrogenated fatty carboxylic acids.

In some embodiments, the composition may be in a form suitable for transdermal administration. These formulations may be prepared, for example, by incorporating the active compound in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, compositions of the invention may further include one or more accessory ingredient(s) selected from encapsulants, diluents, buffers, flavoring agents, binders, disintegrants, surface active agents, thickeners, lubricants, preservatives (including antioxidants), and the like.

In some embodiments, compositions may be formulated for immediate release, sustained release, delayed-onset release or any other release profile known to one skilled in the art.

In some embodiments, the pharmaceutical composition may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target the site of a pathology. For some applications, controlled release formulations obviate the need for frequent dosing to sustain activity at a medically advantageous level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the inhibitor is formulated with appropriate excipients into a pharmaceutical composition that, upon administration, releases the inhibitor in a controlled manner. Examples include single or multiple unit tablet or capsule compositions, oil solutions, suspensions, emulsions, microcapsules, microspheres, molecular complexes, nanoparticles, patches, and liposomes.

In some embodiments, the composition may comprise a “vectorized” form, such as by encapsulation of the inhibitor in a liposome or other encapsulate medium, or by fixation of the inhibitor, e.g., by covalent bonding, chelation, or associative coordination, on a suitable biomolecule, such as those selected from proteins, lipoproteins, glycoproteins, and polysaccharides.

In some embodiments, the composition can be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents. Alternatively, the inhibitor may be incorporated in biocompatible carriers, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Unless the context clearly indicates otherwise, compositions of all embodiments can comprise various pharmaceutically acceptable salts, or other derivatives described previously.

The formulation and preparation of such compositions are well known to those skilled in the art of pharmaceutical formulation. Formulations can be found in Remington: The Science and Practice of Pharmacy.

### Methods

In methods involving administering a combination of an ABCG2 inhibitor and a second pharmaceutically active agent (including chemotherapeutic agents or CNS active agents) the two compounds may be administered together, i.e. at the same time, or at different times, as desired. For example, the ABCG2 inhibitor may be administered before the second pharmaceutically active agent. Likewise, if desired, the ABCG2 inhibitor may be administered before the second pharmaceutically active agent. The most effective order of administration may be readily determined by a clinical practitioner.

The ABCG2 inhibitor and second pharmaceutically active ingredient may be administered in a single composition or separately. The most effective administration may be readily determined by a clinical practitioner, based on routes of administration.

Combinations of ABCG2 inhibitors or combinations of pharmaceutically active agents may be administered.

The compounds or compositions administered may be administered in any of many forms which are well-known to those of skill in the art. For example, they may be administered in any of a variety of art-accepted forms such as tablets, capsules, various injectable formulations, liquids for oral administration and the like, as suitable for the desired means of administration. The preparation which is administered may include one or more than one inhibitory compound, and may further contain other suitable substances and excipients, including but not limited to physiological acceptable buffering agents, stabilizers (e.g. antioxidants), flavoring agents, agents to effect the solubilization of the compound, and the like. Administration of the compounds may be effected by any of a variety of routes that are well-known to those of skill in the art, including but not limited to oral, parenteral, intravenously, via inhalation, and the like. Further, the compounds may be administered in conjunction with other appropriate treatment modalities, for example, with nutritional supplements, agents to reduce symptoms and treatment with other agents.

In some embodiments, the compositions may be administered orally. Administration to human patients or other animals is generally carried out using a physiologically effective amount of a compound of the invention in a physiologically-acceptable carrier. Suitable carriers and their formulation are described, for example, in Remington's Pharmaceutical Sciences by E. W. Martin.

In some embodiments, the compositions may be administered systemically, for example, formulated in a pharmaceutically-acceptable buffer such as physiological saline. Routes of administration include, for example, subcutaneous, intravenous, intraperitoneally, intramuscular, or intradermal injections that provide continuous, sustained levels of the drug in the patient. Administration to human patients or other animals is generally carried out using a physiologically effective amount of a compound of the invention in a physiologically-acceptable carrier. Suitable carriers and their formulation are described, for example, in Remington's Pharmaceutical Sciences by E. W. Martin.

The formulation and preparation of such compositions are well known to those skilled in the art of pharmaceutical formulation. Formulations can be found in Remington: The Science and Practice of Pharmacy.

For example, compositions according to the invention may be provided in a form suitable for administration by sterile injection. To prepare such a composition, the compositions(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

The compositions may be provided in unit dosage forms (e.g., in single-dose ampules), or in vials containing several doses and in which a suitable preservative may be added. A composition of the invention may be in any suitable form such as a solution, a suspension, an emulsion, an infusion device, or a delivery device for implantation or it may be presented as a dry powder to be reconstituted with water or another suitable vehicle before use. The composition may include suitable parenterally acceptable carriers and/or excipients.

The amount of the compound/agent to be administered varies depending upon the manner of administration, the age and body weight of the subject/patient, and with the subject's symptoms and condition. A compound is generally administered at a dosage that best achieves medical goals with the fewest corresponding side effects.

In some embodiments, the compositions including biologically active fragments, variants, or analogs thereof, can be administered by any suitable route including, but not limited to: oral, intracranial, intracerebral, intraventricular, intraperitoneal, intrathecal, intraspinal, topical, rectal, transdermal, subcutaneous, intramuscular, intravenous, intranasal, sub-lingual, mucosal, nasal, ophthalmic, subcutaneous, intramuscular, intravenous, intra-articular, intra-arterial, sub-arachinoid, bronchial, lymphatic, and intra-uterille administration, and other dosage forms for systemic delivery of active ingredients.

Those of skill in the art will recognize that the precise quantity of such a compound to be administered will vary from case to case, and is best determined by a skilled practitioner such as a physician. For example, the amount may vary based on several characteristics of the patient, e.g. age, gender, weight, overall physical condition, extent of disease, and the like. Further, the individual characteristics of the compound itself (e.g. Ki, selectivity, IC₅₀, solubility, bioavailability, and the like) will also play a role in the amount of compound that must be administered. However, in general, the required amount will be such that the concentration of compound in the blood stream of the patient is about equal to or larger than the IC₅₀ or Kᵢ of the compound.

The composition may be administered parenterally by injection, infusion or implantation in dosage forms, formulations, or via suitable delivery devices or implants containing conventional, non-toxic pharmaceutically acceptable carriers and/or adjuvants. In one embodiment, the compositions are added to a retained physiological fluid, such as cerebrospinal fluid, blood, or synovial fluid. The compositions of the invention can be amenable to direct injection, application or infusion at a site of disease or injury.

In one approach, a composition of the invention is provided within an implant, such as an osmotic pump, or in a graft having appropriately transformed cells. Methods of introduction may also be provided by rechargeable or biodegradable devices. Various slow release polymeric devices have been developed and tested for the controlled delivery of drugs, including proteinacious biopharmaceuticals. A variety of biocompatible polymers (including hydrogels), including both biodegradable and non-degradable polymers, can be used to form an implant for the sustained release of a bioactive factor at a particular target site.

### Dosage

The administration of a compound may be by any suitable means that results in a concentration of the compound that, combined with other components, is effective in preventing, diagnosing, prognosing, ameliorating, reducing, or stabilizing a deficit or disorder.

Generally, the amount of administered agent of the invention will be empirically determined in accordance with information and protocols known in the art. Often the relevant amount will be such that the concentration of compound in the blood stream of the patient is about equal to or larger than the IC₅₀ or Kᵢ of the compound. Typically agents are administered in the range of about 10 to 1000 μg/kg of the recipient. Other additives may be included, such as stabilizers, bactericides, and anti-fungals. These additives are present in conventional amounts.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

Terms listed in single tense also include multiple unless the context indicates otherwise.

The above disclosure generally describes exemplary embodiments of the present invention. The examples disclosed below are provided to illustrate the invention but not to limit its scope. Other variants of the invention will be readily apparent to one of ordinary skill in the art and are encompassed by the appended claims. All publications, databases, patents and patent applications disclosed herein are hereby incorporated by reference in their entirety.

### EXAMPLES

### Example 1

### Bioluminescence Imaging (BLI) assay

Reagents. D-Luciferin sodium salt was obtained from Gold Biotechnology, Inc. (St. Louis, Mo.). Verapamil (VP), colchicine (Col), and MTX were purchased from Sigma Chemical Company (St Louis, Mo.). BODIPY-prazosin was obtained from Invitrogen (Carlsbad, Calif.). Glafenine, flavoxate hydrochloride and doxazocin mesylate were obtained from Sigma Chemical Company (St. Louis, Mo.). Fumitremorgin C (FTC) was a kind gift of Dr. S. Bates (National Cancer Institute). All compounds were prepared in dimethylsulfoxide (DMSO) for in vitro experiments. For in vivo experiments, ABCG2 inhibitor was dissolved in ethanol/cremophor EL/saline (1:1:6).

Cell lines. The establishment of ABCG2-overexpressing HEK293 cells stably transfected with CMV-luc2CP/Hygro (referred to here as HEK293/ABCG2/fLuc) has been described previously (Zhang et al., “Hedgehog pathway inhibitor HhAntag691 is a potent inhibitor of ABCG2/BCRP and ABCB1/Pgp,” Neoplasia, vol. 11, no. 1, pp. 96-101, 2009). In brief, HEK293 cells were cultured in minimum essential medium (Invitrogen) supplemented with 10% FBS, and HEK293 cells were stably transfected with ABCG2-expressing construct, maintained in medium containing 1 mg/ml G418. Firefly luciferase-expressing HEK293 cells were established by transient transfection with CMVluc2CP/Hygro, after selection in 50 μg/ml hygromycin B. Transient transfection was performed with FuGENE6 transfection reagent (Roche Pharmaceuticals, Nutly, N.J.) according to the manufacturer's instructions. Control empty vector-transfected HEK293 cells were stably transfected with CMV-Iuc2CP/Hygro in the same way and are referred to here as HEK293/empty/fLuc. Cells were cultured in MEM (Invitrogen, Carlesbad, Calif.) supplemented with 10% FBS, penicillin and streptomycin. HEK293 cells stably transfected with ABCG2-expressing construct were maintained in medium containing 1 mg/mL G418 and 50 μg/mL hygromycin B. ABCG2-overexpressing NCI-H460 human non-small cell lung carcinoma cells (National Cancer Institute, Frederick, Md.) were established and characterized as described previously (Robey et al., “A functional assay for detection of the mitoxantrone resistance protein, MXR (ABCG2),” Biochimica et biophysica acta, vol. 1512, no. 2, pp. 171-182, 2001). They were maintained in RPMI 1640 medium supplemented with 10% FBS, penicillin, and streptomycin. All cultures were maintained at 37° C. in a humidified 5% CO₂/95% air incubator.

Statistic evaluation of the BLI-based assay. The screen was performed in a 96-well format. HEK293/ABCG2/fLuc cells were plated from 1-8×10⁻⁴/well and treated with solvent only or with fumitremorgin C (FTC) as a positive control. D-luciferin concentrations varied from 20-100 μg/mL, and imaging data were acquired every five min for one hr. The quality of this BLI based high-throughput screen assay was evaluated statistically as described previously (Zhang et al., “A Simple Statistical Parameter for Use in Evaluation and Validation of High Throughput Screening Assays,” J Biomol Screen, vol. 4, no. 2, pp. 67-73, 1999). Z′ values were calculated for each combination of parameters. An ideal assay is expected to produce Z′=1, and Z′ values of 1>Z′ 0.5 reflect an excellent assay. The Z′-values obtained from this assay ranged from 0.5 to 0.9.

BLI assay. HEK293/ABCG2/fLuc cells were plated into 96-well plates at a density of 4×10⁴ cells/100 μL per well and were allowed to attach overnight. The following day, 10 μL of each compound or the control solvent was transferred from a compound library in a 96-well, high-throughput format into the wells using a multichannel pipette. The final concentration of each compound was approximately 17 μM. 5 L of D-luciferin (1.2 mg/mL in PBS) were then added to achieve a final concentration of ˜50 μg/mL. The plates were gently tapped to assure that all solutions were well mixed, and imaging commenced immediately. Images were taken every 5 minutes for ˜1 h. Light output from each well was quantified at the 40 min time point after initiation of imaging, and the signal-to-background (S/B) ratio of the light output from each compound divided by that from the control well was calculated. This S/B ratio serves as an indicator of the potency of ABCG2 inhibition, the mechanism by which BLI signal is enhanced.

Assay performance. Signal-to-noise (SN) ratio, signal-to-background (S/B) ratio and Z′ values, which indicate the robustness of the assay, were calculated as described previously (Zhang et al., “A Simple Statistical Parameter for Use in Evaluation and Validation of High Throughput Screening Assays,” J Biomol Screen, vol. 4, no. 2, pp. 67-73, 1999). Background was defined as the light output from cells incubated with D-luciferin and the solvent only.

Screening of the JHCCL using the BLI assay. The JHCCL is composed primarily of compounds approved by the US Food and Drug Administration (FDA) and is the most complete library of clinically-approved drugs (Chong et al., “A clinical drug library screen identifies astemizole as an antimalarial agent,” Nature chemical biology, vol. 2, no. 8, pp. 415-416, 2006; Chong et al., “Identification of type 1 inosine monophosphate dehydrogenase as an antiangiogenic drug target,” Journal of medicinal chemistry, vol. 49, no. 9, pp. 2677-2680, 2006).

Images were taken every 5 min for ˜1 h, and light output from each well at the 40 min time point was chosen for quantification. The SB ratio of the light output from each compound divided by that from the control well was calculated. This ratio was used as an indicator of the potency of ABCG2 inhibition, the mechanism by which BLI signal is enhanced.

The result of the full screen is presented in Table 2. Two hundred and nineteen candidate ABCG2 inhibitors were identified from 3,273 compounds screened. Candidate inhibitors are defined as compounds producing at least two-fold signal enhancement over control values. About 150 weaker inhibitors were also identified. Among the 219 potent (>two-fold signal enhancement) inhibitors, 88 (˜40%) had not been previously reported to be an inhibitor or substrate of any ABC transporter. The majority (−84%) of the ˜150 weak inhibitors had not been previously reported to be either inhibitors or substrates of ABC transporters. Forty seven compounds demonstrated signal enhancement of five-fold. Of those, ten are known ABCG2 inhibitors or substrates (Table 3), validating the assay. The identification of many previously reported ABCG2 inhibitors, including both potent and weak ones, such as gefitinib (Nakamura et al., “Gefitinib (”Iressa“, ZD1839), an epidermal growth factor receptor tyrosine kinase inhibitor, reverses breast cancer resistance protein/ABCG2-mediated drug resistance,” Cancer research, vol. 65, no. 4, pp. 1541-1546, 2005), reserpine (Zhou et al., “The ABC transporter Bcrpl/ABCG2 is expressed in a wide variety of stem cells and is a molecular determinant of the side-population phenotype,” Nature medicine, vol. 7, no. 9, pp. 1028-1034, 2001), dipyridamole (Zhang et al., “BCRP transports dipyridamole and is inhibited by calcium channel blockers,” Pharmaceutical research, vol. 22, no. 12, pp. 2023-2034, 2005), and curcumin Limtrakul et al., “Modulation of function of three ABC drug transporters, P-glycoprotein (ABCB1), mitoxantrone resistance protein (ABCG2) and multidrug resistance protein 1 (ABCC1) by tetrahydrocurcumin, a major metabolite of curcumin,” Molecular and cellular biochemistry, vol. 296, no. 1-2, pp. 85-95, 2007), suggests that the assay is sensitive. The most potent of the novel inhibitors, glafenine, enhanced the BLI signal by ˜20-fold (Table 4).

TABLE 2
All compounds causing BLI enhancement

| Compound | Fold | Therapeutic Effect |
|---|---|---|
| glafenine | 20.6 | analgesic |
| tracazolate | 20.0 | sedative |
| gefitinib | 19.0 | antineoplastic |
| calcimycin | 16.9 | calcium ionophore |
| doxazosin mesylate | 15.9 | antihypertensive |
| verteporfin | 11.3 | opthalmic |
| flavoxate hydrochloride | 11.2 | antispasmodic |
| brij | 11.1 | n/a* |
| quinacrine | 10.6 | anthelminthic |
| grapefruit oil | 10.6 | n/a |
| danthron | 10.2 | cathartic |
| carvedilol | 10.0 | antihypertensive |
| dihydroergotamine mesylate | 9.6 | vasoconstrictor |
| harmine | 8.9 | n/a |
| harmaline | 8.8 | central nervous system stimulant |
| prazosin | 8.4 | antihypertensive |
| clebopride maleate | 7.9 | antiemetic, antispasmodic |
| silver nitrate | 7.7 | antibacterial |
| dipyridamole | 7.1 | antithrombotic |
| myrrh oil | 7.0 | therapeutic plant |
| isorhamnetin | 7.0 | n/a |
| gramicidin | 6.9 | antibacterial |
| clebopride | 6.7 | antiemetic |
| rotenone | 6.7 | acaricide, ectoparasiticide |
| clomiphene citrate | 6.7 | gonad stimulating principle |
| aromatic cascara fluid extract | 6.6 | therapeutic plant |
| sildenafil | 6.6 | erect le dysfunction |
| emodin | 6.4 | antimicrobial, anticancer, |
| | | cathartic |
| flubendazole | 6.3 | anthelminthic |
| curcumin | 6.3 | nutrient |
| metyrapone | 6.3 | diagnostic aid |
| danthron | 6.3 | laxative |
| periciazine | 6.3 | antipsychotic |
| isoreserpine | 6.2 | antihypertensive |
| acepromazine | 6.2 | sedative |
| nelfinavir mesylate | 6.1 | antiviral |
| flutamide | 6.1 | antineoplastic |
| podophyllum resin | 6.1 | dermatologic |
| gambogic acid | 6.0 | antibacterial |
| niguldipine | 5.9 | antihypertensive |
| piperacetazine | 5.8 | antipsychotic |
| digitoxin | 5.7 | antiarrythmic |
| acetophenazine maleate | 5.6 | antipsychotic |
| eupatorin | 5.6 | emetic |
| reserpine | 5.4 | antihypertensive |
| estrone hemisuccinate | 5.4 | estrogen |
| riboflavin | 5.4 | vitamin |
| raloxifene hydrochloride | 5.4 | bone resorption |
| o-dianisidine | 5.0 | n/a |
| hesperetin | 5.0 | antispasmodic |
| oligomycin | 5.0 | antibiotic, antifungal |
| saquinavir mesylate | 4.9 | antiviral |
| orange oil, cold-pressed | 4.7 | therapeutic plant |
| prochlorperazine dimaleate | 4.4 | antiemetic |
| methoxsalen | 4.4 | dermatologic |
| ivermect n | 4.3 | anthelmintic |
| kaempferol | 4.3 | n/a |
| 1,3-dipropyl-8- | 4.3 | A-1 adenosine receptor |
| cyclopentylxanthine [DPCPX] | | antagonist |
| flufenazine hydrochloride | 4.3 | H1 antihistamine |
| citropen | 4.3 | constituent of bergamot oil |
| resiniferatoxin | 4.2 | n/a |
| perphenazine | 4.2 | antipsychotic |
| 13-estradiol | 4.2 | estrogen |
| terfenadine | 4.2 | H1 antihistamine |
| estrone acetate | 4.1 | estrogen |
| lomerizine hydrochloride | 4.0 | antimigraine |
| irinotecan hydrochloride | 4.0 | antineoplastic |
| rosuvastatin | 3.9 | antihyperlipidemic |
| pyrantel | 3.9 | anthelminthic |
| amodiaquin | 3.8 | antimalarial |
| donepezil hydrochloride | 3.8 | nootropic |
| hydroxyitraconazole | 3.8 | n/a |
| cilostazol | 3.8 | antithrombotic |
| pentifylline | 3.8 | vasodilator |
| methyl 7-deshydroxypyrogallin | 3.7 | antioxidant |
| 4-carboxylate | | |
| 2′,4′-dihydroxychalcone 4′- | 3.6 | n/a |
| glucoside | | |
| estradiol cypionate | 3.6 | estrogen |
| nifedipine | 3.6 | antianginal |
| amicinonide | 3.6 | antiinflammatory |
| orange oil | 3.6 | therapeutic plant |
| fluphenazine N-mustard | 3.6 | n/a |
| lopinavir/ritonavir | 3.5 | antiviral (HIV) |
| aclarubicin | 3.5 | antineoplastic |
| atovaquone | 3.5 | antibacterial |
| brimonidine | 3.4 | antiglaucoma |
| khellin | 3.4 | vasodilator |
| clobetasol propionate | 3.3 | glucocorticoid, antiinflammatory |
| praziquantel | 3.3 | anthelminthic |
| hypericin | 3.3 | antidepressant |
| vardenafil | 3.3 | erect le dysfunction |
| nisoldipine | 3.2 | antihypertensive |
| beclomethasone dipropionate | 3.2 | antiinflammatory |
| oltipraz | 3.2 | antiviral |
| calcifediol | 3.2 | bone resportion |
| mebeverine hydrochloride | 3.2 | smooth muscle relaxant |
| alexidine hydrochloride | 3.1 | antibacterial |
| rhein | 3.1 | n/a |
| tegaserod maleate | 3.1 | n/a |
| quinalizarin | 3.0 | indicator |
| amlodipine mesylate | 3.0 | antianginal |
| dolasetron mesylate | 3.0 | antiemetic |
| felodipine | 3.0 | antihypertensive |
| sulmazole | 3.0 | cardiotonic |
| aminacrine | 2.9 | antiseptic |
| β-escin | 2.9 | antihypotensive |
| harmalol hydrochloride | 2.9 | central nervous system stimulant |
| nicardipine | 2.9 | antianginal |
| methoxsalen | 2.9 | antipsoriatic |
| dicyclomine hydrochloride | 2.9 | anticholinergic |
| 8-cyclopentyltheophylline | 2.9 | adenosine agonist |
| flunarizine hydrochloride | 2.9 | vasodilator |
| hydroxyflutamine | 2.9 | antineoplastic |
| croton oil | 2.8 | laxative |
| metochalcone | 2.8 | choleretic, diuretic |
| astemizole | 2.8 | antihistaminic |
| stanozolol | 2.8 | stero d |
| phenazine methosulfate | 2.8 | n/a |
| nimodipine | 2.8 | vasodilator |
| 6,7-dihydroxyflavone | 2.7 | antihaemorrhagic |
| ondansetron hydrochloride | 2.7 | antiemetic |
| pravadoline | 2.7 | analgesic |
| simvastatin | 2.7 | antihyperlipidemic |
| juniper tar | 2.7 | therapeutic plant |
| lasalocid sodium | 2.7 | antibiotic |
| estradiol propionate | 2.6 | estrogen |
| diperodon hydrochloride | 2.6 | anesthetic |
| ethaverine hydrochloride | 2.6 | antispasmodic |
| thiethylperazine malate | 2.6 | antiemetic |
| betamethasone valerate | 2.6 | glucocorticoid |
| nefazodone hydrochloride | 2.6 | antidepressant |
| ceftazidime | 2.5 | antibiotic |
| ellipticine | 2.5 | n/a |
| aklavine hydrochloride | 2.5 | antibiotic |
| econazole | 2.5 | antifungal |
| hycanthone | 2.5 | anthelminthic |
| origanum oil | 2.5 | therapeutic plant |
| prasterone (DHEA) | 2.5 | stero d |
| domperidone | 2.5 | antiemetic, dopam ne antagonist |
| chlorocresol | 2.5 | topical antiseptic |
| cycloleucylglycine | 2.5 | antinarcotic |
| th oridaz ne | 2.5 | antipsychotic |
| verapamil hydrochloride | 2.4 | adrenegic receptor blocker, |
| | | calcium channel blocker |
| chloroxylenol | 2.4 | antibacterial |
| 2-(2,6-dimethoxyphenoxyethyl) | 2.4 | n/a |
| aminomethy1-1,4-benzodioxane | | |
| hydrochloride | | |
| naftopidil | 2.4 | antihypertensive |
| diclazuril | 2.4 | antibacterial |
| azelastine hydrochloride | 2.4 | antihistaminic |
| salmeterol xinafoate | 2.4 | bronchodilator |
| ritonavir | 2.4 | antiviral |
| gallopamil | 2.4 | antianginal |
| propafenone | 2.4 | antiarrhythmic |
| ethinyl estradiol | 2.4 | estrogen |
| birch tar oil rectified | 2.4 | therapeutic plant |
| medrysone | 2.3 | glucocorticoid |
| fenofibrate | 2.3 | antihyperlipidemic |
| chrysin | 2.3 | diuretic |
| casein enzymatic hydrolysate | 2.3 | nutrient |
| moricizine hydrochloride | 2.3 | antiarrhythmic |
| cyclosporin A | 2.3 | immunosuppressant |
| toremifene | 2.3 | antineoplastic |
| noscapine hydrochloride | 2.3 | antitussive |
| antimycin | 2.3 | antifungal, antiviral |
| dexamethasone acetate | 2.2 | antiinflammatory |
| granisetron hydrochloride | 2.2 | antiemetic |
| floxuridine | 2.2 | antineoplastic, antimetabolite |
| halcinonide | 2.2 | antiinflammatory |
| celecoxib | 2.2 | antiarthritic, cyclooxygenase-2 |
| | | inhibitor |
| periciazine | 2.2 | antipsychotic |
| 3-formyl rifamycin | 2.2 | antibacterial |
| itopride hydrochloride | 2.2 | n/a |
| pimecrol mus | 2.2 | immunosuppressant |
| mercaptamine hydrochloride | 2.2 | depigmentation, radiation |
| | | protectant |
| benzethonium chloride | 2.2 | anti-infective |
| salicylanilide | 2.2 | antifungal |
| berberine bisulfate | 2.2 | antiprotozoal |
| coal tar | 2.1 | therapeutic plant |
| prochlorperazine dimaleate | 2.1 | antiemetic |
| cepharanthine | 2.1 | antineoplastic |
| comiferin | 2.1 | antioxidant |
| tolperisone hydrochloride | 2.1 | skeletal muscle relaxant |
| methylbenzethonium chloride | 2.1 | antiseptic |
| hesperidin methyl chalcone | 2.1 | therapeutic plant |
| chlorzoxazone | 2.1 | skeletal muscle relaxant |
| exemestane | 2.1 | antineoplastic |
| phenylbutyric acid | 2.1 | anti-inflammatory |
| megestrol acetate | 2.1 | progestogen |
| sulconazole | 2.1 | antifungal |
| oxfendazole | 2.1 | anthelminthic |
| physcion | 2.1 | antimicrobial, cathartic |
| bromocriptine mesylate | 2.1 | prolactin inhibitor, |
| | | antiparkinsonian |
| pimozide | 2.0 | antipsychotic |
| quindine sulfate dihydrate | 2.0 | antimalarial |
| cyproterone | 2.0 | antiandrogen |
| lemongrass oil | 2.0 | therapeutic plant |
| racecadotril | 2.0 | antidiarrheal |
| telmisartan | 2.0 | antihypertensive |
| cimicifugin | 2.0 | therapeutic plant |
| papaverine hydrochloride | 2.0 | smooth muscle relaxant, |
| | | cerebral vasodilator |
| 9-amino-1,2,3,4- | 2.0 | anticholinesterase |
| tetrahydroacridine | | |
| hydrochloride | | |
| fluphenazine | 2.0 | antipsychotic |
| nicergoline | 2.0 | vasodilator |
| benztropin Methane- | 2.0 | antiparkinsonian |
| difluprednate | 2.0 | anti-inflammatory |
| oxiconazole nitrate | 2.0 | antifungal |

*No therapeutic effect available

TABLE 3
Known compounds with five-fold or greater BLI enhancement.

| Compound | Fold | Known therapeutic effect |
|---|---|---|
| gefitinib (40) | 19.0 | antineoplastic |
| harmine (41) | 8.9 | n/a* |
| prazosin (42) | 8.4 | antihypertensive |
| dipyridamole (18) | 7.1 | antithrombotic |
| curcumin (43) | 6.3 | nutrient |
| nelfinavir mesylate (38) | 6.1 | antiviral |
| niguldipine (44) | 5.9 | antihypertensive |
| riboflavin (45) | 5.4 | antispasmodic |
| reserpine (4) | 5.4 | antihypertensive |
| hesperetin (46) | 5.0 | antispasmodic |

*No therapeutic effect available

TABLE 4
Previously unknown compounds with five-
fold or greater BLI enhancement.

| Compound | Fold | Known therapeutic Effect |
|---|---|---|
| glafenine | 20.6 | analgesic |
| tracazolate | 20 | sedative |
| calcimycin (A23187) | 16.9 | calcium ionophore |
| doxazosin mesylate salt | 15.9 | antihypertensive |
| verteporfin | 11.3 | ophthalmic |
| flavoxate hydrochloride | 11.2 | antispasmodic |
| Brij 30 | 11.1 | n/a |
| quinacrine | 10.6 | anthelmintic |
| grapefruit oil | 10.6 | n/a |
| dihydroergotamine mesylate | 9.6 | vasoconstrictor, specific |
| | | in migraine |
| harmaline | 8.8 | CNS stimulant, antiparkinsonian |
| clebopride maleate | 7.9 | antiemetic, antispasmodic |
| silver nitrate | 7.7 | antibacterial |
| isorhamnetin | 7.0 | n/a |
| gramicidin A | 6.9 | antibacterial |
| clebopride | 6.7 | antiemetic |
| rotenone | 6.7 | acaricide, ectoparasiticide, |
| | | inhibits NADH2 oxidation to NAD |
| clomiphene citrate | 6.7 | gonad stimulating principle |
| aromatic cascara fluid extract | 6.6 | therapeutic plant |
| sildenafil | 6.6 | impotency therapy |
| emodin | 6.4 | antimicrobial, anticancer, cathartic |
| flubendazole | 6.3 | anthelminthic |
| metyrapone (2-methy1-1,2-di- | 6.3 | diagnostic aid |
| periciazine (propericiazine) | 6.3 | antipsychotic |
| isoreserpine | 6.2 | antihypertensive |
| acepromazine | 6.2 | sedative |
| flutamide | 6.1 | antineoplastic |
| podophyllum resin | 6.1 | dermatologic |
| gambogic acid | 6.0 | antibacterial, inhibit Hela cell |
| | | growth in vitro |
| piperacetazine | 5.8 | antipsychotic |
| digitoxin | 5.7 | cardiotonic, cardiotoxic; inhibits |
| | | Na+/K+ ATPase |
| acetophenazine maleate | 5.6 | antipsychotic |
| eupatorin | 5.6 | emetic ex Eupatorium spp and |
| | | other Compositae |
| estrone hemisuccinate | 5.4 | estrogen |
| raloxifene hydrochloride | 5.4 | bone resorption |
| o-dianisidine | 5.0 | not approved |
| oligomycin | 5.0 | antibiotic, antifungal |

Sensitivity of the BLI assay. The BLI assay was further evaluated by searching the library for previously known ABCG2 inhibitors. Due to the relatively recent characterization of ABCG2, relatively few ABCG2 inhibitors are known (Ahmed-Belkacem et al., “Inhibitors of cancer cell multidrug resistance mediated by breast cancer resistance protein (BCRP/ABCG2),” Anti-cancer drugs, vol. 17, no. 3, pp. 239-243, 2006). Twenty five previously known ABCG2 inhibitors/substrates were found to be included in the HDL. In addition to the ten compounds listed in Table 3 producing significant BLI signal, fifteen additional, known ABCG2 inhibitors are present in the HDL (Table 5). Twenty two of those compounds enhanced the BLI signal significantly (from 2.3- to 19-fold), and only three, naringenin, acacetin and genistein, enhanced the BLI signal less than two-fold (1.9-, 1.8- and 1.2-fold, respectively).

TABLE 5
Additional previously known ABCG2 Inhibitors.

| Compound | fold | Reference |
|---|---|---|
| estrone | 4.1 | 1 |
| estradiol | 3.6 | 1 |
| 6,7-dihydroxyflavone | 2.7 | 1 |
| chrysin | 2.3 | 1 |
| naringenin | 1.9 | 1 |
| acacetin | 1.8 | 1 |
| genistein | 1.2 | 1 |
| nifedipine | 3.6 | 2 |
| nicardipine | 2.9 | 2 |
| saquinavir mesylate | 4.9 | 3 |
| lopinavir/ritonavir | 3.5 | 3 |
| dipyridamole | 7.1 | 4 |
| nicardipine | 2.9 | 4 |
| nimodipine | 2.8 | 4 |
| cyclosporine A | 2.3 | 5 |

1 - Robey et al., Cancer metastasis reviews, vol. 26, no. 1, pp. 39-57, 2007.
2 - Zhou et al., Drug metabolism and disposition: the biological fate of chemicals, vol. 33, no. 8, pp. 1220-1228, 2005.
3 - Weiss et al., The Journal of antimicrobial chemotherapy, vol. 59, no. 2, pp. 238-245, 2007.
4 - Zhang et al., Pharmaceutical research, vol. 22, no. 12, pp. 2023-2034, 2005.
5 - Gupta et al., Cancer chemotherapy and pharmacology, vol. 58, no. 3, pp. 374-83, 2006.

### Example 2

### Mitoxantrone (MTX) Resensitization Assay

The ABC transporter-inhibiting ability of the potential inhibitors identified were further tested by evaluating their ability to resensitize ABCG2-overexpressing NCI-H460/MX20 cells to MTX, or MDCKII cells overexpressing Pgp or MRP1, to Col. Cells were plated in 96-well plates at 1×10⁴ per well and allowed to attach. MTX was added to 15 μM or 30 μM, with or without a putative ABCG2 inhibitor. Colchicine was added at 1 μM for MDCKII/Pgp cells and 0.3 μM for MDCKII/MRP1 cells. After two days of incubation cell viability was assessed using the XTT assay as described previously (Zhang et al., “Hedgehog pathway inhibitor HhAntag691 is a potent inhibitor of ABCG2/BCRP and ABCB1/Pgp,” Neoplasia, vol. 11, no. 1, pp. 96-101, 2009). In brief, 1 mg/ml XTT (Polysciences, Warrington, Pa.) was mixed with 0.025 mM PMS (Sigma), and 50 μl of the mixture was added to each well and incubated for 4 hours at 37° C. After the plates were mixed on a plate shaker, absorbance at 450 nm was measured. All results were normalized to a percentage of absorbance obtained in controls.

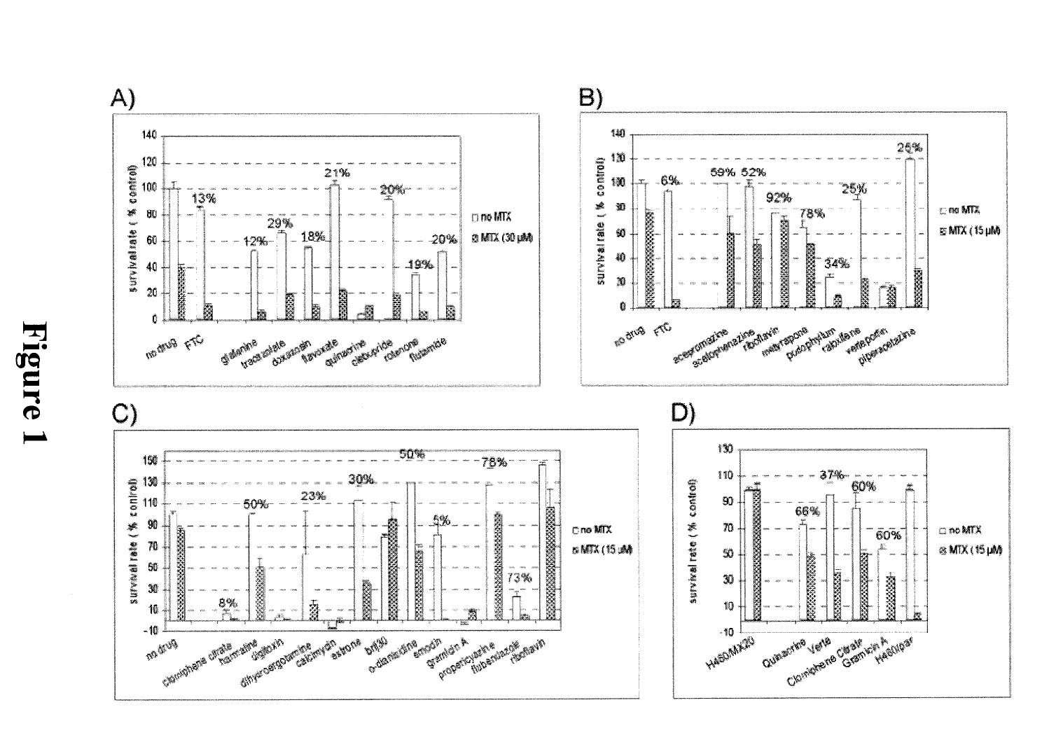

Twenty-eight novel candidate ABCG2 inhibitors identified in the BLI screen were tested by the MTX resensitization, a hallmark of ABCG2 inhibitor function (Robey et al., “ABCG2: determining its relevance in clinical drug resistance,” Cancer metastasis reviews, vol. 26, no. 1, pp. 39-57, 2007). Both ABCG2 overexpressing H460/MX20 cells and the parent line were treated with MTX (15 or 30 μM) for three days. As expected, H460/MX20 cells survived exposure to MTX better than the parent cells due to the induced expression of ABCG2 (˜40% vs. ˜9% survival in 30 μM MTX, ˜80% vs <20% in 15 μM MTX). The potent, selective ABCG2 inhibitor FTC restored the sensitivity of H460/MX20 cells to MTX and significantly reduced their survival rate. Twenty-eight novel candidate inhibitors were initially tested at 20 μM for three days. Twenty demonstrated a similar capacity to sensitize H460/MX20 cells to MTX, confirming that they are indeed ABCG2 inhibitors (FIG. 1A, 1B and 1C). Brij 30 was found to resensitize H460/MX20 cells to MTX significantly after two days of incubation (data not shown). The most active inhibitors were glafenine and doxazosin mesylate, which, at concentrations of 20 μM, reduced the survival of H460/MX20 cells to 13% and 18%, respectively. These results were consistent with their considerable activity in the BLI screen (20- and 16-fold signal enhancement, respectively). That suggests that the magnitude of BLI signal enhancement can reflect the potency of ABCG2 inhibitors.

Six compounds, including quinacrine, verteporfin, digitoxin, clomiphene citrate, calcimycin and gramicin A, were too cytotoxic to be tested at 20 μM (FIG. 1A, 1B and 1C). Each was tested again at 1 μM with 15 μM MTX for two days. At this lower concentration, quinacrine, verteporfin, clomiphene citrate, and gramicin A showed resensitization of H460/MX20 cells to MTX, confirming them as ABCG2 inhibitors (FIG. 1D). The other two, digitoxin and calcimycin, were tested at even lower concentrations (0.3, 0.1 and 0.03 μM). They were no longer cytotoxic at 0.1 and 0.03 μM, but did not reduce the survival rate of H460/MX20 cells significantly after two days when co-incubated with 15 μM MTX (data not shown). However, at these lower concentrations (0.03 and 0.1 μM), they enhance BLI signal minimally.

In summary, 26 of the 28 candidate compounds identified were confirmed by the MTX resensitization assay to be new ABCG2 inhibitors. The false positive rate is low, with false positive compounds difficult to test in the MTX resensitization assay by virtue of their direct cytotoxicity.

### Example 3

### BODIPY-prazosin Uptake Assay

ABCG2-overexpressing HEK293 cells were plated in 6- well plates at a density of 1.1×10⁶ cells per well and were allowed to attach. Cells were then changed into medium containing 0.25 μM BODIPY-prazosin (Robey, et al, “Mutations at amino-acid 482 in the ABCG2 gene affect substrate and antagonist specificity,” British journal of cancer, vol. 89, no. 10, pp. 1971-1978, 2003), and compound to be tested was added to a final concentration of 20 μM, followed by incubation at 37° C. for 1 h. Cells were then harvested, washed with ice-cold PBS once, resuspended in cold PBS, and analysed with flow cytometry. Analyses were performed with FACScan (Becton Dickinson, Fullerton, Calif.) with an excitation wavelength of 488 nm and an emission wavelength of 530 nm. Ten thousand events were counted per sample. The resultant histograms were analyzed with CellQuest software (Becton Dickinson).

Data analysis. Livinglmage (Xenogen Corp., Alameda, Calif.) and IGOR (Wavemetrics, Lake Oswego, Oreg.) image analysis software were used to superimpose and analyze the corresponding gray scale photographs and false color BLI images. Light intensities of regions of interest (ROls) were expressed as total flux (photons/sec). The IC₅₀ values of identified ABCG2 inhibitors were calculated using GraphPad Prism version 4.0 for Windows (GraphPad Software, San Diego, Calif.) using variable-slope logistic nonlinear regression analysis. Data are presented as mean+SEM, n=3.

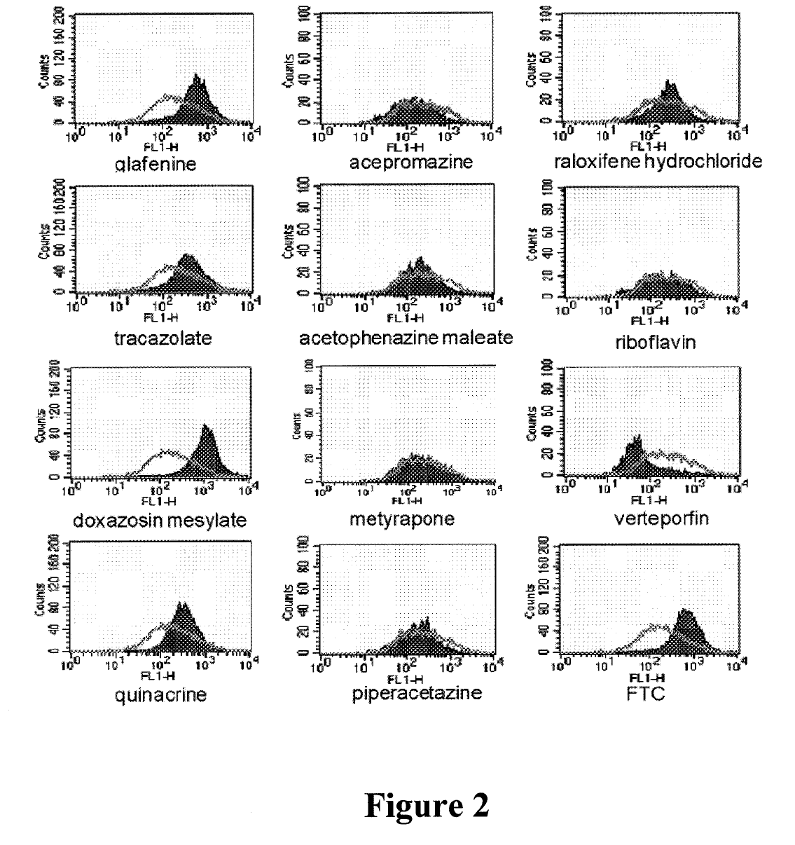

Sixteen of the candidate ABCG2 inhibitors were also tested with the BODIPY-prazosin assay. HEK293/ABCG2 cells were incubated with BODIPY-prazosin and each test compound, and then subjected to flow cytometry. Nine of the 16 compounds, glafenine, tracazolate, doxazosin mesylate, quinacrine, clebopride, flutamide, flavoxate hydrochloride, rotenone, and podophyllum resin were positive by this assay. Notably, seven compounds identified by the BLI assay, acepromazine, acetophenazine maleate, metyrapone, piperacetazine, raloxifene hydrochloride, riboflavin and verteporfin were negative according to this assay (FIG. 2). Among these seven, six were confirmed by the MTX resensitization assay. Verteporfin was among the compounds too cytotoxic to validate by MTX assay. The results of the MTX resensitization and the BODIPY-prazosin assays are compared in Table 6. Quinacrine, although too cytotoxic to be tested in the MTX assay, was confirmed as an ABCG2 inhibitor by the BODIPY-prazosin uptake assay.

TABLE 6
Results of the mitoxantrone (MTX) resensitization assay*
and the BODIPY-prazosin dye uptake (BP) assays.

| Compound | MTX | BP |
|---|---|---|
| glafenine | Y | Y |
| tracazolate | Y | Y |
| doxazosin mesylate | Y | Y |
| verteporfin | Y | N |
| flavoxate hydrochloride | Y | Y |
| quinacrine | Y | Y |
| clebopride maleate | Y | Y |
| metyrapone | Y | N |
| rotenone | Y | Y |
| acepromazine | Y | N |
| flutamide | Y | Y |
| podophyllum resin | Y | Y |
| piperacetazine | Y | N |
| acetophenazine maleate | Y | N |
| raloxifene hydrochloride | Y | N |
| riboflavin | Y | N |

*Y = activity confirmed, N = activity not confirmed

### Example 4

### In vivo Bioluminescence Imaging

Animal protocols were approved by the Johns Hopkins University Animal Care and Use Committee. Both HEK293/ABCG2/fLuc and HEK293/empty/ABCG2 cells were implanted subcutaneously into 6-week-old female nude mice at 1×10⁶ cells at each site. The IVIS 200 small animal imaging system (Xenogen Corp., Alameda, Calif.) was used for BLI and 2.5% isoflurane was used for anesthesia. D-luciferin was injected intraperitoneally (i.p.) into mice at 150 mg/kg, and imaging was performed every few minutes for more than 1 h. ABCG2 inhibitor was administered via tail vein injection as a bolus during imaging, with imaging continued thereafter.

Data analysis. Livinglmage (Xenogen Corp.) and IGOR (Wavemetrics, Lake Oswego, Oreg.) image analysis software were used to superimpose and analyze the corresponding gray scale photographs and false color BLI images. Light intensities of regions of interest (ROls) were expressed as total flux (photons/sec). The IC₅₀ values of identified ABCG2 inhibitors were calculated using GraphPad Prism version 4.0 for Windows (GraphPad Software, San Diego, Calif.) using variable-slope logistic nonlinear regression analysis. Data are presented as mean±SEM, n=3.

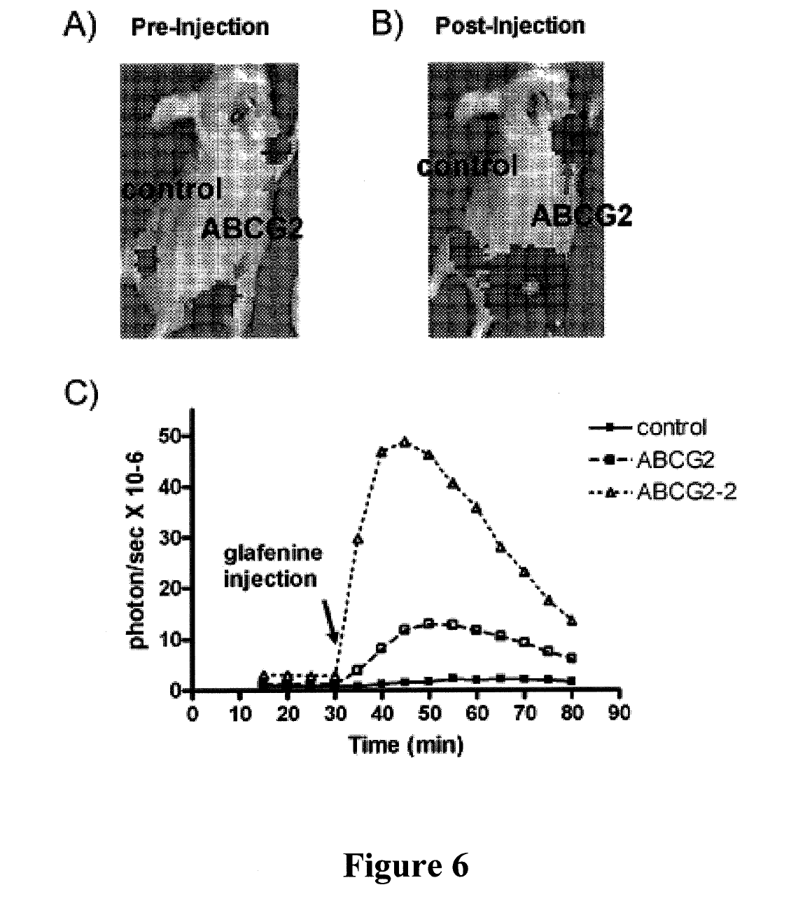

In vivo inhibition of ABCG2 activity by selected new ABCG2 inhibitors. Two of the newly identified ABCG2 inhibitors, glafenine and doxazosin mesylate, were tested further for their ability to inhibit ABCG2 function in vivo. We have previously shown that administration of FTC in vivo can significantly enhance D-luciferin-dependent BLI signal output of xenografts derived from ABCG2-overexpressing HEK293 cells (Zhang et al, “ABCG2/BCRP expression modulates D-Luciferin based bioluminescence imaging,” Cancer research, vol. 67, no. 19, pp. 9389-9397, 2007). Here we used the same strategy to test the effect of these new ABCG2 inhibitors in vivo. HEK293/empty/fLuc and HEK293/ABCG2/fLuc cells were implanted subcutaneously into opposite flanks of female nude mice. Five mice were implanted to generate ten ABCG2-overexpressing xenografts and five controls. Animals were imaged after D-luciferin administration, which was followed by a bolus injection of a single dose of ABCG2 inhibitor and continued imaging. After glafenine injection (25 mg/kg i.v.), nine out of 10 ABCG2-overexpressing xenografts showed enhanced BLI signal over the control in the same mouse. Those 10 xenografts showed an average of ˜2.9-fold signal enhancement over the control with the highest approaching 6.7- fold (FIG. 6). Glafenine caused increases in BLI signal of up to ˜11.6- and ˜17.4-fold in two separate HEK293/ABCG2/fLuc xenografts (right front and rear flanks) in the same mouse compared to the signals generated by those xenografts immediately before injection. By contrast, the BLI signal of the HEK293/empty/fLuc xenograft in the left flank increased by only ˜2.6-fold (FIG. 6). Doxazosin mesylate injection caused a similar but weaker BLI signal enhancement of ABCG2-overexpressing xenografts in vivo (data not shown).

### Example 5

### IC₅₀ Determination

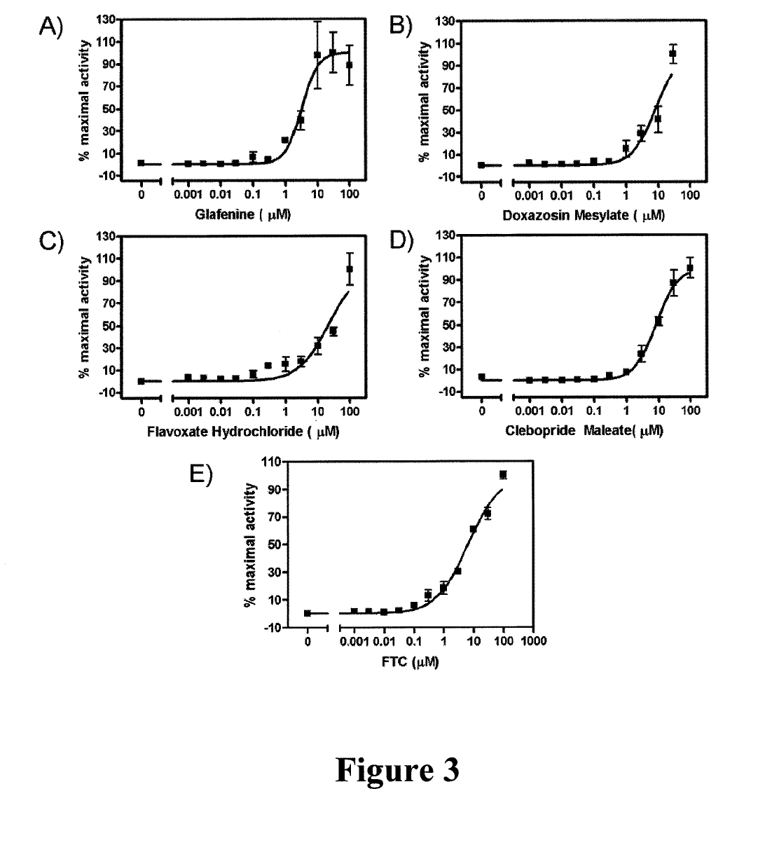

An ABCG2 inhibitor can enhance fLuc-based BLI signal in a dose-dependent manner, as discussed previously. The BLI signal-enhancing effect of selected ABCG2 inhibitors was evaluated within the range of 0.001 μM -100 μM, with HEK293/ABCG2/fLuc cells and 50 μg/mL D-Iuciferin. The data obtained at 40 min after imaging commencement were chosen arbitrarily to be plotted (FIG. 3). The IC₅₀ value of glafenine as an ABCG2 inhibitor was calculated to be 3.2 μM. For three other ABCG2 inhibitors, doxazosin mesylate, flavoxate hydrochloride, and clebopride maleate, the BLI signal did not reach a plateau, even at concentrations as high as 100 μM. Assuming that the BLI signal produced by each compound at 100 μM approaches a maximum value, the IC₅₀ values of doxazosin mesylate, flavoxate hydrochloride, and clebopride maleate can be calculated to be 8.0 μM, 20 μM, and 8.2 μM, respectively. The same assay was used to calculate the IC₅₀ value of FTC, and it was determined to be 6.6 μM using the 30 min data. Although that value deviates from the IC₅₀ values reported for FTC in literature (0.3˜1.3 μM), the discrepancy may be caused by the fact that the assays involve different substrates. In terms of its ability to inhibit ATPase, Robey et al. measured the IC₅₀ value of FTC to be 1 μM (13), while Özvegy et al. obtained values of 1.3 μM (21) and 0.4 μM (22). The IC₅₀ value of FTC was also reported to be 0.8 μM using the pheophorbide A fluorescent dye uptake assay (23). According to those previous reports, FTC reached the plateau of its ABCG2-inhibiting effect at a concentration of 10 μM, but the BLI assay indicates that higher concentrations would be needed to provide a maximal inhibitory effect (FIG. 3E).

### Example 6

### MTX Resensitization Dose Dependency

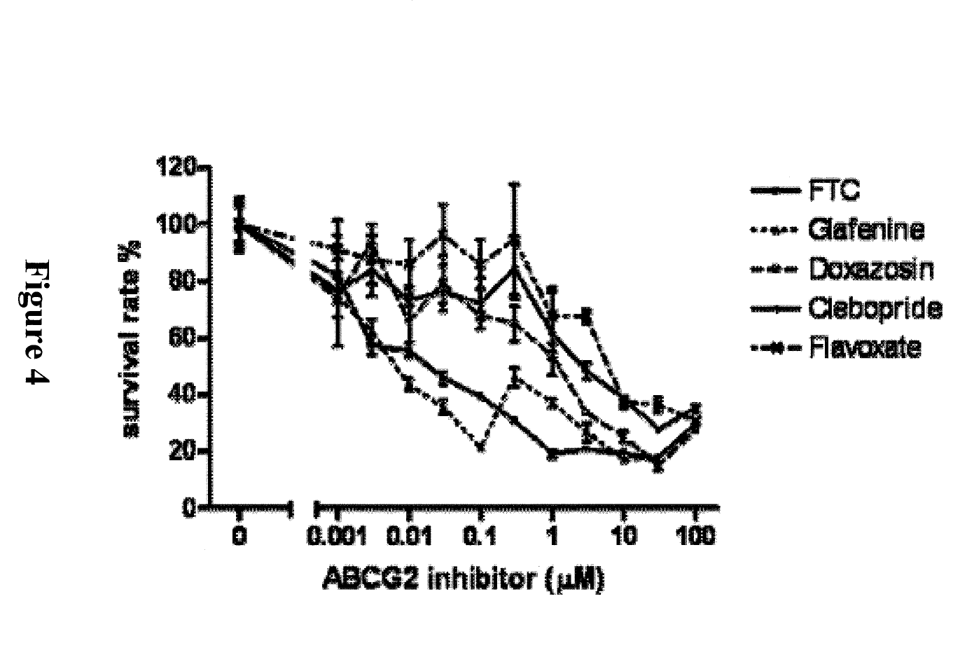

The dose-dependent effect of ABCG2 inhibitors was also evaluated with the MTX resensitization assay. ABCG2 overexpressing H460/MX20 cells were incubated for three days with increasing concentrations of each ABCG2 inhibitor in addition to 15 μM MTX, and the survival rates were plotted against the concentration of each compound (FIG. 4). Consistent with the IC₅₀ values of each ABCG2 inhibitor obtained from BLI assay, glafenine proved a more potent ABCG2 inhibitor than FTC, doxazosin mesylate, clebopride maleate, and flavoxate hydrochloride.

### Example 6

### ABCG2 Specificity

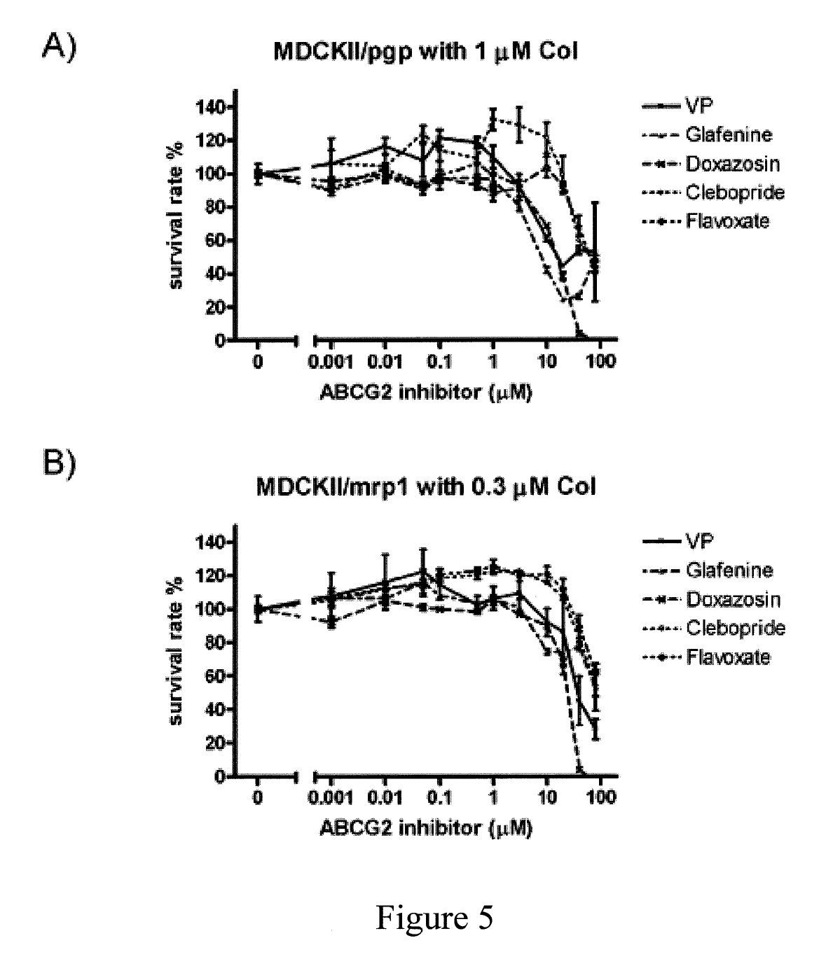

To check whether newly identified ABCG2 inhibitors were specific to ABCG2 as opposed to inhibiting other MDR pumps generally, they were also tested for their ability to inhibit ABCB1/Pgp (P-glycoprotein) and ABCC1/MRP1 (Multiple Drug Resistance Protein 1). The resensitization assay was performed with MDCKII cells overexpressing Pgp or MRP1 (Evers et al., “Inhibitory effect of the reversal agents V-104, GF120918 and Pluronic L61 on MDR1 Pgp-, MRP1- and MRP2-mediated transport,” British journal of cancer, vol. 83, no. 3, pp. 366-374, 2000) using colchicine (Col), a Pgp and MRP1 substrate (Ambudkar et al., “Biochemical, cellular, and pharmacological aspects of the multidrug transporter,” Annual review of pharmacology and toxicology, vol. 39, pp. 361-398, 1999; Shen et al., “Multiple drug-resistant human KB carcinoma cells independently selected for high-level resistance to colchicine, adriamycin, or vinblastine show changes in expression of specific proteins,” The Journal of biological chemistry, vol. 261, no. 17, pp. 7762-7770, 1986). MDCKII cells overexpressing Pgp or MRP1 were incubated for two days in medium containing 1 μM (for Pgp) or 0.3 μM (for MRP1) Col and increasing concentrations of each ABCG2 inhibitor. As shown in FIG. 5A, compared to Verapamil (VP), glafenine is a more potent Pgp inhibitor, doxazosin mesylate has similar potency, and clebopride maleate and flavoxate hydrochloride demonstrate weak Pgp-inhibiting ability at relatively high concentration (30 μM). Glafenine and doxazosin mesylate have similar potencies to VP for MRP1 inhibition, while clebopride maleate and flavoxate hydrochloride proved weak, even at relatively high concentration (30 μM) (FIG. 5B). However, all of these ABCG2 inhibitors are specific for ABCG2 at low concentrations (1 μM). For example, glafenine can effectively resensitize H460/MX20 cells to MTX at a concentration as low as 0.001 μM (FIG. 4), but does not provide resensitization of MDCKII/Pgp or MDCKII/MRP1 cells to Col until 1 μM or 10 μM, respectively.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the following claims that such claims be interpreted as broadly as is reasonable.

1. A method for identifying inhibitors of ABCG2 comprising
imaging at least one culture comprising a test compound, luciferin and cells that express ABCG2 and firefly luciferase; and
selecting a test compound resulting in at least two-fold bioluminescence signal enchancement as an ABCG2 inhibitor.

2. The method of claim 1, wherein the culture is prepared by adding test compound to a culture comprising cells that express ABCG2 and firefly luciferase.

3. The method of claim 1, further comprising imaging cultures comprising different concentrations of the same test compound.

4. The method of claim 1, further comprising confirming the activity of the ABCG2 inhibitor.

5. The method of claim 4, wherein confirming the activity comprises a resensitization assay or a dye uptake assay.

6. A composition comprising an ABCG2 inhibitor identified by the method of claim 1, a chemotherapeutic agent and a pharmaceutically acceptable carrier or excipient, wherein the ABCG2 inhibitor and the chemotherapeutic agent in combination are present in a therapeutically effective amount and the therapeutic effectiveness of the chemotherapeutic agent in the combination with the ABCG2 inhibitor is greater than the therapeutic effectiveness of the chemotherapeutic agent in the absence of the ABCG2 inhibitor.

7. A composition comprising an ABCG2 inhibitor identified by the method of claim 1, a CNS active agent, and a pharmaceutically acceptable carrier or excipient, wherein the ABCG2 inhibitor and the CNS active agent are present in a therapeutically effective amount and the therapeutic effectiveness of the CNS active agent in combination with the ABCG2 inhibitor is greater than the therapeutic effectiveness of the CNS agent in the absence of the ABCG2 inhibitor.

8. A method of treating a cellular proliferative disorder comprising administering to a patient in need of treatment an ABCG2 inhibitor identified by the method of claim 1, optionally together with an additional chemotherapeutic agent.

9. (canceled)

10. A method of treating a central nervous system disorder comprising administering to a patient in need of treatment an ABCG2 inhibitor identified by the method of claim 1 and a therapeutically effective amount of a CNS active agent.

11. A method of treating a multiple drug resistant tumor comprising administering an ABCG2 inhibitor identified by the method of claim 1 and a therapeutically effective amount of a chemotherapeutic agent different from the ABCG2 inhibitor.

12. A method of treating a cellular proliferative disorder comprising administering to a patient in need of treatment a therapeutic amount of a compound selected from the group consisting of the compounds of Tables 2-6, optionally together with a therapeutically effective amount of a chemotherapeutic agent different from the ABCG2 inhibitor.

13. A method according to claim 12, wherein the compound is selected from the group consisting of the compounds of table 2, but excluding the compounds of tables 3 and 5.

14. A method according to claim 12, wherein the compound is selected from the group consisting of glafenine, tracazolate, calcimycin (A23187), doxazosin, verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil, emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof.

15. A method according to claim 12, wherein the compound is selected from the group consisting of doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof.

16. A method according to claim 12, wherein the compound is selected from the group consisting of Doxazosin, flavoxate, dihydroergotamine, and combinations thereof.

17-21. (canceled)

22. A method of imaging cells, tumors, tissues or organs that express ABCG2 comprising administering an effective amount of an ABCG2 inhibitor identified by the method of claim 1, labeled with one or more radioisotopes.

23. A method of imaging cells, tumors, tissues or organs that express ABCG2 comprising administering an effective amount of an ABCG2 inhibitor selected from the group consisting of the compounds listed in Tables 2-6, labeled with one or more radioisotopes.

24. (canceled)

25. A method according to claim 23, wherein the compound is selected from the group consisting of glafenine, tracazolate, calcimycin (A23187), doxazosin, verteporfin, flavoxate, Brij 30, quinacrine, grapefruit oil, dihydroergotamine, harmaline, clebopride, silver nitrate isorhamnetin, gramicidin A, clebopride, rotenone, clomiphene, aromatic cascara fluid extract, sildenafil, emodin, flubendazole, metyrapone (2-methyl-1,2-dipyridin-3-yl-propan-1-one), periciazine (propericiazine), isoreserpine, acepromazine, flutamide, podophyllum resin, gambogic acid, piperacetazine, digitoxin, acetophenazine maleate, eupatorin, estrone hemisuccinate, raloxifene hydrochloride, o-dianisidine, oligomycin and combinations thereof.

26. A method according to claim 23, wherein the compound is selected from the group consisting of doxazosin, Clebopride, Rotenone, Flavoxate, Dihydroergotamine, Glafenine, Flutamide, Emodin, Clomiphene, Flubendazole, Raloxifene, Piperacetazine, Tracazolate, Estrone, Podophyllum resin, Harmaline, o-Dianisidine, Acetophenazine, Acepromazine, Metyrapone, propericyazine, and combinations thereof.

27. A method according to claim 23, wherein the compound is selected from the group consisting of Doxazosin, flavoxate, dihydroergotamine, and combinations thereof.
